(19) United States
(12) Patent Application Publication
(10) Pub. No.: US 2014/0150138 A1
(43) Pub. Date: May 29, 2014

(54) Novel Proteinase Inhibitor Promotes Resistance to Insects

(71) Applicant: United States as Represented by the Secretary of, (US)

(72) Inventor: Anna C. Smigocki, Silver Spring, MD (US)

(73) Assignee: United States as Represented by the Secretary of Agriculture, Washington, DC (US)

(21) Appl. No.: 13/685,017

(22) Filed: Nov. 26, 2012

(51) Int. Cl. C07K 14/81; C12N 15/82; C07H 21/04

(52) U.S. Cl. CPC C07K 14/811; C07H 21/04; C12N 15/8286
USPC 800/302; 536/23.6; 530/379; 536/24.1; 435/320.1; 435/419

(57) ABSTRACT

A novel Beta vulgaris serine proteinase inhibitor gene (BvSTI) and its protein are identified in response to insect feeding on B. vulgaris seedlings. BvSTI is cloned into an expression vector with constitutive promoter and transformed into Nicotiana benthamiana plants to assess BvSTI's ability to impart resistance to lepidopteran insect pests. A reporter gene GUS is also cloned into an expression vector under control of the BvSTI gene promoter and transformed into N. benthamiana plants to determine if the promoter induces expression of the gene upon wounding and insect feeding. BvSTI DNA and amino acid sequences and the promoter sequences from various strains of B. vulgaris are obtained. Transformation of BvSTI cDNA under control of constitutive promoter or an inducible promoter into economically valuable plants is useful for effective control of insect pests that feed on the economically valuable plants and utilize serine proteases for digestion.

### BACKGROUND OF THE INVENTION

1. Field of Invention

This invention relates to a novel serine proteinase inhibitor gene, BvSTI and the protein encoded by BvSTI. This invention also relates to expression vectors, plants, and seeds containing BvSTI and/or the protein encoded by BvSTI. This invention also relates to the method of enhancing a plant's resistance to certain insect pests by the expression of the BvSTI gene or the presence of BvSTI protein in the plant. BvSTI promoters useful for the expression of BvSTI and other polynucleotides in plants are included in this invention.

2. Description of the Relevant Art

Assimilation of dietary proteins is critical to normal insect growth and development. Insect digestive proteases are grouped into several mechanistic classes based on the amino acid residue or metal ion that is involved in peptide bond catalysis. Major midgut proteases of the Lepidoptera and Diptera insect orders tend to be predominately of the serine (trypsin) type (Matsumoto et al. 1995. Eur. J. Biochem. 27:582-587; Pendola and Greenberg, 1975. Ann. Entomol. Soc. Am. 68 (2):341-345; Srinivasan et al. 2006. Cell Mol. Bio. Letters 11:132-154; Wilhite et al. 2000. Exp. Appl. 97:229-233). The trypsin type serine proteases, which include chymotrypsin- and elastase-like serine protease, often are major midgut proteolytic enzymes in lepidopteran insects (Jongsma et al. 1996. Trends in Biotechnology 14: 331-333; Lara et al. 2000. Transgenic Research 9:169-178; Srinivasan et al. 2006). In the Homoptera and Coleoptera orders, major proteases utilized for digestion tend to be of the cysteine class. These proteases are targeted by many naturally occurring plant proteinase inhibitors that are characterized by their specificity toward proteases (Abe et al. 1994. J. Biochem. 116:489-492; Brzin et al. 1998. L. Plant Sci. 2:17-26; Christeller et al. 1998. Eur. J. Biochem. 254:160-167; Jongsma & Bolter, 1997. J. Insect Physiol. 43:885-895).

Inhibition of insects' digestive proteolytic enzymes is a desirable target for development of effective strategies to control insect pests. Proteinase inhibitors' significant role in plants' natural defense mechanisms against insects has been well-documented (Fan and Wu 2005. Bot. Bull. Acad. Sin. 46:273-292; Lawrence and Koundal 2002. Electron. J. Biotechnol. 5(1):93-102; Ussuf et al. 2001. Curr. Sci. 80(7):847-853). Defensive capacities of plant proteinase inhibitors rely on inhibition of the insect's digestive proteases thus limiting the availability of amino acids necessary for normal insect growth and development (De Leo et al. 2002. Nucleic Acids Res. 30(1):347-348).

Via recombinant DNA technology, one can transfer a proteinase inhibitor gene from one plant to other plants and enhance the other plants' insect resistance level. Over-expression of heterologous proteinase inhibitor genes in transgenic plants significantly reduce or inhibit larval growth and feeding on the transgenic plants (Abdeen et al. 2005. Plant Mol. Biol. 57:189-202; Boulter et al. 1990. Crop Protection 9:351-354; Charity et al. 2005. Function Plant Biol. 32:35-44; Cowgill et al. 2002. Mol. Ecol. 11:821-827; Delledonne et al. 2001. Mol. Breed 7:35-42; Duan et al. 1996. Nature Biotech. 14:494-498; Graham et al. 1997. Ann. Appl. Biol. 131:133-139; Maheswaran et al. 2007. Plant Cell Rep. 26:773-782; Mehlo et al. 2005. Proc. Nat. Acad. Sci. 102:7812-7816; Ninkovic et al. 2007, Plant Cell Tiss. Organ Cult. 91:289-294; Samac and Smigocki, 2003. Phytopath. 93 (7):799-804; Schüter et al. 2010. J. Exp. Bot. 61(15):4169-4183; Telang et al. 2003. Phytochem. 63(6):643-652). Expression of bitter gourd proteinase inhibitors in transgenic plants result in a greater than 80% reduction of Helicoverpa armigera serine proteases activity while feeding on the transgenic plants (Telang et al. 2003). Similarly, expression of rice cysteine proteinase inhibitor genes, oryzacystatin I and II, in transgenic plants increase the transgenic plant's resistance to several coleopteran pests, as well as nematodes, that commonly use cysteine proteases for protein digestion (Schlüter et al. 2010; Pandey and Jamal, 2010. Int. J. Biotech. Biochem. 6(4):513-520; Ninković et al. 2007. Plant Cell Tiss. Organ Cult. 91:289-294; Samac and Smigocki, 2003; Urwin et al. 1995. Plant J. 8:121-131; Kondo et al. 1990. FEBS Lett. 278:87-90; Abe and Arai, 1985. Agric. Biol. Chem. 49:3349-3350). Conversely, suppression of proteinase inhibitor gene expression in transgenic potato results in an increase in larval weights of Colorado potato beetle (Leptinotarsa decemlineata) and beet armyworm (Spodoptera exigua) (Ortego et al. 2001. J. Insect Physiol. 47(11):1291-1300).

One major challenge of the proteinase inhibitor based insect control strategy is the management of the inherent and induced complexity of the insect gut proteases. Because non-targeted proteases may compensate for the blocked proteases, several approaches are needed to combat this problem. One solution to this problem is gene stacking, or expression of multiple proteinase inhibitors in a transgenic plant. Gene stacking includes, for example, using multiple protein inhibitors (either same or different class of proteinase inhibitors) obtained from different plants as well as using multiple proteinase inhibitors (either same or different class of proteinase inhibitors) from the same plant. In the broadest terms, gene stacking can include a transgenic plant having multiple DNA sequences encoding desired proteins for expression, regardless of the function of the desired proteins. The DNA sequences can encode proteins that impart resistance to herbicides, or proteins that inhibit enzymes (e.g., proteinase inhibitors), or enzymes that are useful for biosynthetic production of a desired substance, or proteins that improve the plant in some other fashion. For example, expression of tobacco and potato inhibitors of the same class simultaneously in the transgenic plant is effective in increasing insect resistance (Dunse et al., 2010. Proc. Natl. Acad. Sci. 107(34):15011-15015). Further, expression in tomato of two different classes of potato proteinase inhibitor genes is effective for control of both a lepidopteran and a dipteran insect (Abdeen et al. 2005). The potential to control more than one pest by gene stacking makes the proteinase inhibitor approach highly desirable for plant improvement. Yet, because of the variety of the insect pests and their ability to use multiple proteases to overcome the effects of one proteinase inhibitor, there is a need to discover new proteinase inhibitor genes and add the new proteinase inhibitor genes to plants to improve the plant's resistance to insects. Proteinase inhibitors such as those derived from non-host plants to which the insect has had minimal or no prior exposure may prove most useful for enhancing insect resistance in transgenic plants.

### SUMMARY OF THE INVENTION

It is an object of this invention to have a novel serine proteinase inhibitor, BvSTI, obtained from sugar beets, and to have the polynucleotide sequence and amino acid sequence of the novel serine proteinase inhibitor. Polynucleotide and amino acid sequences that are at least 95%, at least 90%, or at least 85% identical to the DNA or amino acid sequence of this novel serine proteinase inhibitor are included in this invention. BvSTI is obtained from various varieties of sugar beets.

Novel promoters for BvSTI is also an object of this invention. The novel promoters are obtained from various varieties of sugar beets. These novel promoters induce the transcription of BvSTI after the plant is wounded by an insect.

It is another object of this invention to have expression vectors that contain polynucleotides which encode BvSTI and polypeptides that are at least 95%, at least 90%, or at least 85% identical to the sequence of BvSTI. It is a further object of this invention that the expression vectors contain constitutive promoters or inducible promoters that control the transcription of the BvSTI sequences contained in these expression vectors. These expression vectors can also contain the inducible BvSTI promoters obtained in the present invention which induce expression of BvSTI after an insect wounds the plant.

It is an object of this invention to have transgenic plants which contain polynucleotides which encode BvSTI and polypeptides that are at least 95%, at least 90%, or at least 85% identical to the sequence of BvSTI. These transgenic plants contain the expression vectors of the present invention which have BvSTI or polynucleotides that are at least 95%, at least 90%, or at least 85% identical to the sequence of BvSTI under control of constitutive or inducible promoters. The inducible promoters could be the BvSTI promoters of the present invention. It is a further object of this invention that the transgenic plants include transgenic plant cells and transgenic plant seeds. It is another object of this invention that the transgenic plants are economically valuable plants and can be either monocots or dicots.

It is another object of this invention to have a method of increasing a plant's resistance to insects which utilize serine protease in digestion by generating a transgenic plant by transfecting the plant with a polynucleotide encoding BvSTI under control of an inducible or constitutive promoter. It is a further object that the inducible promoter is a BvSTI promoter. Another object of this invention is that the transgenic plant is an economically valuable plant. The polynucleotide encoding BvSTI for the present invention can be at least 95%, at least 90%, or at least 85% identical to the DNA sequence of BvSTI. Alternatively, the polynucleotide encoding BvSTI for the present invention can encode a protein that is at least 95%, at least 90%, or at least 85% identical to the amino acid sequence of BvSTI.

### BRIEF DESCRIPTION OF THE DRAWINGS

FIG. 1 is the amino acid sequence alignment of BvSTI EST with proteinase inhibitors, Mcp20 (GenBank access number BAB82379.1), trypsin (GenBank access number NP_{—}001237952.1), and Kunitz (GenBank access number NP_{—}001237716.1).

FIG. 2 is a schematic of pBvSTI. RB, right border; LB, left border; p35S, cauliflower mosaic virus (CaMV) 35S promoter; hpt, hygromycin phosphotransferase selectable marker gene; NdeI restriction enzyme sites; arrows indicate direction of transcription from the p35S promoter. Horizontal bar indicates the 400-bp fragment of the BvsTI gene used as a probe for Southern blots.

FIG. 3 is a schematic representation of the expression vector pCAMBIA 1301 plasmid T-DNA regions with GUS uidA gene driven by either sugar beet BvSTI promoter (pBvSTIpro-GUS) or CaMV 35S promoter (p35S-GUS). T-DNA fragment in both vectors also contains between left (LB) and right (RB) borders selectable hygromycin phosphotransferase gene (hptII) under control of CaMV 35S promoter and multiple cloning site (pUC18MCS).

FIG. 4 is an alignment of the BvSTI promoter DNA sequences obtained from genomic DNA from various B. vulgaris strains and red beet (USDA accession PI179180).

FIG. 5 is an alignment of the DNA sequence of BvSTI obtained from the indicated strains of B. vulgaris and red beet (USDA accession PI179180).

### DETAILED DESCRIPTION OF THE INVENTION

Sugar beet (Beta vulgaris) is an important food crop, being one of only two plant sources from which sugar is economically produced. Grown in temperate regions of the world, the large succulent taproots of sugar beet are processed into crystalline sucrose that accounts for 35% of global raw sugar production (Oerke and Dehne 2004. Crop Prot. 23:275-285; Smith 1987. Fehr WR (ed) Principles of Cultivar Development: Crop Species, Vol 2. MacMillan Publishing Company, NY, pp 577-625). Planted in the spring and harvested in the autumn of the same year the rosette leaves and the white fleshy taproots are attacked by numerous pests and pathogens that reduce yields by up to 80% (Jafari et al. 2009. Euphytica 165(2):333-344; Zhang et al. 2008. Ann. Appl. Biol. 152:143-156; Oerke and Dehne 2004; Allen et al. 1985. Appl. Environ. Microbiol. 50(5):1123-1127). Pesticides are only partially effective; they reduce yield losses by approximately 26% (Oerke and Dehne 2004). Targeted alteration of crop genotypes aimed to enhance pest tolerance, mostly by reducing the reproductive rate of a pest, through conventional breeding has produced undesirable effects. Some of these effects, which include reduction of yields, are caused by the transfer of undesirable traits along with the traits of interest. The root yield of an insect resistant breeding line, F1015, was 25% less than the root yield of commercial hybrids (Campbell et al. 2000. Crop Sci. 40:867-868). To reduce these negative effects, biotechnological approaches have provided an alternate strategy for germplasm improvement of many important crops (Lemaux 2008. Annu. Rev. Plant Biol. 59:771-812; Moose and Mumm 2008. Plant Physiol. 147:969-977). Continued success of biotechnology, however, hinges on the availability of well characterized beneficial genes often derived from valuable germplasm used in breeding programs.

The most destructive insect pest of sugar beet in North America is the sugar beet root maggot (Tetanops myopaeformis Roder). Sugar beet root maggots are found in more than half of all North American sugar beet acreage and cause seedling wilt and death, secondary root growth, reduced taproot size and secondary pathogen invasions, all leading to significant crop damage and yield loss. To date, only three sugar beet lines, F1016, F1015 and F1024, with moderate but incomplete levels of resistance to sugar beet root maggots have been released for use in sugar beet improvement programs (Campbell et al. 2000; Campbell et al. 2010. J. Plant Registry 5(2):241-247).

To identify sugar beet DNA loci important in insect resistance, sugar beet root maggots are fed on sugar beet lines F1016 and F1010. An analysis of the genes that are up-regulated reveals approximately one-hundred fifty genes. Out of these approximately one-hundred fifty genes, one gene, BvSTI, is determined to be useful to providing resistance to sugar beet root maggots and other insect pests which utilize serine proteases to digest food. BvSTI encodes a Kunitz-type serine proteinase inhibitor belonging to a class of proteinase inhibitors that are involved in hydrolytic deactivation of trypsin.

This novel serine proteinase inhibitor gene, BvSTI, and the protein encoded, BvSTI, are useful for imparting resistance to economically valuable plants against Lepidoptera, Diptera, and other insects that utilize serine proteases for digestion. BvSTI inhibits the hydrolytic activity of trypsin proteases in insects containing serine protease in their mid-gut. BvSTI may be used in plants by itself or in combination with other proteinase inhibitors (via gene stacking) to impart resistance to the transgenetic plants against Lepidoptera, Diptera, and other insect orders that utilize serine proteases. The amino acid sequence of BvSTI and homologs are one aspect of the invention. The nucleotide sequence of BvSTI and homologs are another aspect of this invention. Expression vectors containing these nucleotide sequences, as well as transgenic economically valuable plants containing these expression vectors which contain these polynucleotide sequences are included in this invention. Transgenic plants, including seeds, cells, leaves, and other parts of the transgenic plants, containing BvSTI or BvSTI, are included in this invention.

As used herein, the terms “nucleotides”, “nucleic acid molecule”, “nucleic acid sequence”, “polynucleotide”, polynucleotide sequence”, “oligonucleotide”, “nucleic acid fragment”, “isolated nucleic acid fragment” are used interchangeably herein. These terms encompass nucleotide sequences and the like. A polynucleotide may be a polymer of RNA or DNA that is single- or double-stranded and that optionally contains synthetic, non-natural or altered nucleotide bases. A polynucleotide in the form of a polymer of DNA may contain one or more segments of cDNA, genomic DNA, synthetic DNA, or mixtures thereof. A gene is composed of nucleotides that encode a protein or structural RNA. Usually, an oligonucleotide is shorter than a polynucleotide.

Any expression vector containing the polynucleotides described herein operably linked to a promoter is also covered by this invention. A polynucleotide sequence is operably linked to an expression control sequence(s) (e.g., a promoter and, optionally, an enhancer) when the expression control sequence controls and regulates the transcription and translation of that polynucleotide sequence. An expression vector is a replicon, such as plasmid, phage or cosmid, and which contains the desired polynucleotide sequence operably linked to the expression control sequence(s). The promoter may be, or is identical to, a viral, phage, bacterial, yeast, insect, plant, or mammalian promoter. Similarly, the enhancer may be the sequences of an enhancer from virus, phage, bacteria, yeast, insects, plants, or mammals.

The term “operably linked” refers to the association of two or more nucleic acid fragments on a single polynucleotide so that the function of one is affected by the other. For example, a promoter is operably linked with a coding sequence so that the promoter is capable of affecting the expression of that coding sequence (i.e., that the coding sequence is under the transcriptional control of the promoter). Coding sequences can be operably linked to regulatory sequences in sense or antisense orientation. When a promoter is operably linked to a polynucleotide sequence encoding a protein or polypeptide, the polynucleotide sequence should have an appropriate start signal (e.g., ATG) in front of the polynucleotide sequence to be expressed. Further, the sequences should be in the correct reading frame to permit transcription of the polynucleotide sequence under the control of the expression control sequence and, translation of the desired polypeptide or protein encoded by the polynucleotide sequence. If a gene or polynucleotide sequence that one desires to insert into an expression vector does not contain an appropriate start signal, such a start signal can be inserted in front of the gene or polynucleotide sequence. In addition, a promoter can be operably linked to a RNA gene encoding a functional RNA.

As used herein, the term “express” or “expression” is defined to mean transcription alone. A regulatory element (promoters and optionally an enhancer) is operably linked to the coding sequence of the gene BvSTI such that the regulatory element is capable of controlling the expression of BvSTI. “Altered levels” or “altered expression” refers to the production of gene product(s) in transgenic organisms in amounts or proportions that differ from that of normal or non-transformed organisms.

As used herein, the terms “encoding”, “coding”, or “encoded” when used in the context of a specified polynucleotide mean that the polynucleotide sequence contains the requisite information to guide translation of the nucleotide sequence into a specified protein. The information by which a protein is encoded is specified by the use of codons. A nucleic acid encoding a protein may contain non-translated sequences (e.g., introns) within translated regions of the nucleic acid or may lack such intervening non-translated sequences (e.g., as in cDNA).

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

The present invention also covers polynucleotide sequences which are promoters, more specifically, inducible promoters. A “promoter” is an expression control sequence and is capable of controlling the expression of a coding sequence or functional RNA. In general, a coding sequence is located 3′ to a promoter sequence. The promoter sequence comprises of proximal and more distal upstream elements, the latter elements often referred to as enhancers. Accordingly, an “enhancer” is a nucleotide sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, or even synthetic nucleotide segments. It is understood by those skilled in the art that different promoters may direct the expression of a gene in different tissues or cell types, or at different stages of development, or in response to different environmental conditions. Promoters that cause a polynucleotide to be expressed in most cell types at most times are commonly referred to as “constitutive promoters”. “Inducible promoters” are promoters that cause a polynucleotide to be expressed under specific conditions such as, but not limited to, in specific tissue, at specific stages of development, or in response to specific environmental conditions, e.g., wounding of tissue or presence or absence of a particular compound. New promoters of various types useful in plant cells are constantly being discovered; numerous examples may be found in the compilation by Okamuro and Goldberg. 1989. Biochemistry of Plants 15:1-82. It is further recognized that because in most cases the exact boundaries of regulatory sequences have not been completely defined, nucleic acid fragments of different lengths may have identical promoter activity.

The “translation leader sequence” refers to a nucleotide sequence located between the promoter sequence and the coding sequence. The translation leader sequence is present in the fully processed mRNA upstream of the translation start sequence (ATG). The translation leader sequence may affect processing of the primary transcript to mRNA, mRNA stability or translation efficiency.

The “3′ non-coding sequences” refer to nucleotide sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complementary copy of the DNA sequence, it is referred to as the primary transcript; or it may be an RNA sequence derived from post-transcriptional processing of the primary transcript and is referred to as the mature RNA. “Messenger RNA” or “mRNA” refers to the RNA that is without introns and that can be translated into polypeptides by the cell. “cDNA” refers to a DNA that is complementary to and derived from an mRNA template. The cDNA can be single-stranded or converted to double stranded form using, for example, the Klenow fragment of DNA polymerase I. “Sense” RNA refers to an RNA transcript that includes the mRNA and so can be translated into a polypeptide by the cell. “Antisense”, when used in the context of a particular nucleotide sequence, refers to the complementary strand of the reference transcription product. “Antisense RNA” refers to an RNA transcript that is complementary to all or part of a target primary transcript or mRNA and that blocks the expression of a target gene. The complementarity of an antisense RNA may be with any part of the specific nucleotide sequence, i.e., at the 5′ non-coding sequence, 3′ non-coding sequence, introns, or the coding sequence. “Functional RNA” refers to sense RNA, antisense RNA, ribozymal RNA (rRNA), transfer RNA (tRNA), micro RNA (miRNA), or other RNA that may not be translated but yet has an effect on cellular processes.

“Transformation”, “transgenic”, and “transfection” refers to the transfer of a polynucleotide into the genome of a host organism, resulting in genetically stable inheritance. Such genetically stable inheritance may potentially require the transgenic organism to be subject for a period of time to one or more conditions which require the transcription of some or all of transferred polynucleotide in order for the transgenic organism to live and/or grow. Host organisms containing the transformed polynucleotide are referred to as “transgenic” or “transformed” organisms or “transformants”. Examples of methods of plant transformation include Agrobacterium-mediated transformation (De Blaere et al. 1987. Meth. Enzymol. 143:277) and particle-accelerated or “gene gun” transformation technology (Klein et al. 1987. Nature 327:70-73; U.S. Pat. No. 4,945,050, incorporated herein by reference). Additional transformation methods are disclosed below. Transgenic, transformed, and transformant also refer to any cell, cell line, callus, tissue, plant part, or plant the genotype of which has been altered by the presence of a heterologous polynucleotide including those transgenics, transformed, or transformants initially so altered (first generation or T1) as well as those created by sexual crosses or asexual propagation from the initial transgenic (second or more generation or T2 or higher). “Transgenic”, “transformed” and “transformant” do not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. The expression vector that is used to generate a transgenic organism may integrate into the genome of the transgenic organism or an organelle within the transgenic organism and is no longer a separate replicon.

Isolated polynucleotides of the present invention can be incorporated into recombinant constructs, typically DNA constructs, capable of introduction into and replication in a host cell. Such a construct can be an expression vector that includes a replication system and sequences that are capable of transcription and translation of a polypeptide-encoding sequence in a given host cell. A number of expression vectors suitable for stable transfection of plant cells or for the establishment of transgenic plants have been described in, e.g., Pouwels et al. 1985. Supp. 1987. Cloning Vectors: A Laboratory Manual; Weissbach and Weissbach. 1989. Methods for Plant Molecular Biology, Academic Press, New York; and Flevin et al. 1990. Plant Molecular Biology Manual, Kluwer Academic Publishers, Boston. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive expression), a transcription initiation start site (ATG codon), a ribosome binding site, an RNA processing signal, a transcription termination site, and/or a polyadenylation signal.

A “protein” or “polypeptide” is a chain of amino acids arranged in a specific order determined by the coding sequence in a polynucleotide encoding the protein or polypeptide. Each protein or polypeptide has a unique function.

As used herein, “substantially similar” refers to polynucleotides wherein changes in one or more nucleotide bases results in substitution of one or more amino acids, but do not affect the functional properties of the polypeptide encoded by the nucleotide sequence. In addition, a substantially similar polynucleotide can have one or more nucleotide base pairs different from the reference polynucleotide sequence but still have the identical amino acid sequence of the reference polypeptide because of the degenerate nature of the coding sequence of DNA and RNA (i.e., more than one codon can encode the same amino acid). “Substantially similar” also refers to modifications of the polynucleotides of the instant invention such as deletion or insertion of nucleotides that do not substantially affect the functional properties of the resulting transcript. It is therefore understood that the invention encompasses more than the specific exemplary nucleotide or amino acid sequences and includes functional equivalents thereof which are substantially similar to the exemplary nucleotides or amino acid sequences. Alterations in a polynucleotide that result in the production of a chemically equivalent amino acid at a given site, but do not affect the functional properties of the encoded polypeptide, are well known in the art. Thus, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine or histidine, can also be expected to produce a functionally equivalent protein or polypeptide. Nucleotide changes which result in alteration of the N-terminal and C-terminal portions of the polypeptide would also not be expected to alter the activity of the polypeptide. Each of the proposed modifications is well within the routine skill in the art, as is determination of retention of biological activity of the encoded products. A method of selecting an isolated polynucleotide that affects the level of expression of a polypeptide in a virus or in a host cell (eukaryotic, such as plant, yeast, fungi, or algae; prokaryotic, such as bacteria) may include the steps of: constructing an isolated polynucleotide of the present invention; introducing the isolated polynucleotide into a host cell; measuring the level of a polypeptide in the host cell containing the isolated polynucleotide; and comparing the level of a polypeptide in the host cell containing the isolated polynucleotide with the level of a polypeptide in a host cell that does not contain the isolated polynucleotide.

Moreover, substantially similar nucleic acid fragments may also be characterized by their ability to hybridize. Estimates of such homology are provided by either DNA-DNA or DNA-RNA hybridization under conditions of stringency as is well understood by those skilled in the art (1985. Nucleic Acid Hybridization, Hames and Higgins, Eds., IRL Press, Oxford, U.K.). Stringency conditions can be adjusted to screen for moderately similar fragments, such as homologous sequences from distantly related organisms, to highly similar fragments, such as genes that duplicate functional enzymes from closely related organisms.

Thus, isolated polynucleotide sequences that encode a BvSTI polypeptide and which hybridize under stringent conditions to the BvSTI polynucleotide sequences disclosed herein, or to fragments thereof, are encompassed by the present invention.

Substantially similar nucleic acid fragments of the present invention may also be characterized by the percent identity of the amino acid sequences that they encode to the amino acid sequences disclosed herein, as determined by algorithms commonly employed by those skilled in this art. Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988. CABIOS 4:11-17), the local homology algorithm of Smith et al. (1981. Adv. Appl. Math. 2:482); the homology alignment algorithm of Needleman and Wunsch (1970. J. Mol. Biol. 48:443-453); the search-for-similarity-method of Pearson and Lipman (1988. Proc. Natl. Acad. Sci. 85:2444-2448; the algorithm of Karlin and Altschul (1990. Proc. Natl. Acad. Sci. 87:2264), modified as in Karlin and Altschul (1993. Proc. Natl. Acad. Sci. 90:5873-5877). Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule.

As used herein, “percentage of sequence identity” means the value determined by comparing two optimally aligned sequences over a comparison window, wherein the portion of the polynucleotide sequence in the comparison window may contain additions or deletions (i.e., gaps) as compared to the reference sequence (which does not contain additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

The term “substantial identity” of polynucleotide sequences means that a polynucleotide containing s a sequence that has at least 80% sequence identity, at least 85%, at least 90%, at least 95% sequence identity, or at least 97% sequence identity compared to a reference sequence using one of the alignment programs described above using standard parameters. One of ordinary skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 80%, at least 85%, at least 90%, at least 95%, and at least 97%. Optimal alignment may be conducted using the homology alignment algorithm of Needleman et al. (1970. J. Mol. Biol. 48:443).

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein.

A “substantial portion” of an amino acid or nucleotide sequence is an amino acid or a nucleotide sequence that is sufficient to afford putative identification of the protein or gene that the amino acid or nucleotide sequence contains. Amino acid and nucleotide sequences can be evaluated either manually by one skilled in the art, or by using computer-based sequence comparison and identification tools that employ algorithms such as BLAST. In general, a sequence of approximately ten or more contiguous amino acids or approximately thirty or more contiguous nucleotides is necessary in order to putatively identify a polypeptide or nucleic acid sequence as homologous to a known protein or gene. Moreover, with respect to nucleotide sequences, gene-specific oligonucleotide probes comprising approximately thirty or more contiguous nucleotides may be used in sequence-dependent methods of gene identification and isolation. In addition, short oligonucleotides of approximately twelve or more nucleotides may be use as amplification primers (or “primers”) in PCR in order to obtain a particular nucleic acid fragment comprising the primers. Accordingly, a “substantial portion” of a nucleotide sequence is a nucleotide sequence that will afford specific identification and/or isolation of a nucleic acid fragment comprising the sequence. The instant specification teaches amino acid and nucleotide sequences encoding polypeptides that contain a particular plant protein. The skilled artisan, having the benefit of the sequences as reported herein, may now use all or a substantial portion of the disclosed sequences for purposes known to those skilled in this art. Thus, such a portion represents a “substantial portion” and can be used to establish “substantial identity”, i.e., sequence identity of at least 80%, compared to the reference sequence. Accordingly, the instant invention includes the complete sequences as reported herein as well as substantial portions at those sequences as defined above.

Fragments and variants of the disclosed nucleotide sequences and proteins encoded thereby are also encompassed by the present invention. A “fragment” is a portion of the polynucleotide sequence or a portion of the amino acid sequence and hence protein encoded thereby. Fragments of a polynucleotide sequence may encode protein fragments (polypeptides) that retain the biological activity of the native protein and hence have BvSTI-like activity. Alternatively, fragments of a polynucleotide sequence that are useful as hybridization probes may not encode fragment proteins retaining biological activity.

The term “variant” refers to substantially similar sequences compared to the reference protein, polypeptide, oligonucleotide, or polynucleotide. For nucleotide sequences, conservative variants include those sequences that, because of the degeneracy of the genetic code, encode the amino acid sequence of one of the BvSTI polypeptides of the invention. Naturally occurring allelic variants such as these can be identified with the use of well-known molecular biology techniques, as, for example, with polymerase chain reaction (PCR), a technique used for the amplification of specific DNA segments. Generally, variants of a particular nucleotide sequence of the invention will have generally at least about 85%, at least about 90%, at least about 95% and at least about 97% sequence identity to that particular nucleotide sequence as determined by sequence alignment programs described elsewhere herein.

As used herein, a variant protein means a protein derived from the native protein by deletion, truncation, or addition of one or more amino acids to the N-terminal and/or C-terminal end of the native protein; deletion or addition of one or more amino acids at one or more sites in the native protein; or substitution of one or more amino acids at one or more sites in the native protein. Variant proteins encompassed by the present invention are biologically active; they possess the desired biological activity of the native protein. Variant proteins may result from, for example, genetic polymorphism or from human manipulation. Biologically active variant proteins of a native BvSTI protein of the invention will have at least about 85%, at least about 90%, at least about 95%, and at least about 97% sequence identity to the amino acid sequence for the native protein as determined by sequence alignment programs described herein. A biologically active variant of a protein of the invention may differ from the reference protein by as few as 2-15 amino acid residues, or even 1 amino acid residue.

The polypeptides of the invention may be altered in various ways including amino acid substitutions, deletions, truncations, and insertions. Novel proteins having properties of interest may be created by combining elements and fragments of proteins of the present invention, as well as with other proteins. Methods for such manipulations are generally known in the art. Thus, the genes and nucleotide sequences of the invention include both the naturally occurring sequences as well as mutant forms. Likewise, the proteins of the invention encompass naturally occurring proteins as well as variations and modified forms thereof. Such variant proteins will continue to possess the desired BvSTI activity. Obviously, the mutations that will be made in the DNA encoding the variant must not place the sequence out of reading frame and preferably will not create complementary regions that could produce secondary mRNA structure.

The deletions, truncations, insertions, and substitutions of the protein sequences encompassed herein are not expected to produce radical changes in the characteristics of the protein. However, when it is difficult to predict the exact effect of the substitution, truncation, deletion, or insertion in advance of doing so, one skilled in the art will appreciate that the effect will be evaluated by routine screening assays where the effects of BvSTI protein can be observed.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein.

As used herein, the term “plant” includes reference to whole plants, plant organs (e.g., leaves, stems, roots, etc.), seeds, plant cells, and progeny of same. Parts of transgenic plants are to be understood within the scope of the invention to be, for example, plant cells, protoplasts, tissues, callus, embryos as well as flowers, stems, fruits, leaves, roots originating in transgenic plants or their progeny previously transformed with a DNA molecule of the invention and therefore consisting at least in part of transgenic cells, are also an object of the present invention.

As used herein, the term “plant cell” includes, without limitation, seeds, suspension cultures, embryos, meristematic regions, callus tissue, leaves, roots, shoots, gametophytes, sporophytes, pollen, and micro spores. The class of plants that can be used in the methods of the invention is generally as broad as the class of higher plants amenable to transformation techniques, including both monocotyledonous and dicotyledonous plants (also referred to as monocots and dicots). The inventions described herein can be used in any plant which is a food source for Lepidoptera insects, Diptera insects and any other insects that utilize serine proteases for digestion. Non-limiting examples of such plants include cotton, maize (corn), peanut, sunflower, tobacco, rice, wheat, rye, barley, alfalfa, tomato, cucumber, soya, sweet potato, grapes, rapeseed, sugar beet, tea, strawberry, rose, chrysanthemum, poplar, eggplant, pepper, walnut, pistachio, mango, banana, potato, carrot, celery, parsley, conifers (which are neither monocots nor dicots), citrus (oranges, lemons, grapefruit and the like), lilies, orchids, onions, asparagus, palm, cauliflower, cabbage, broccoli, turnips, soybean, pea, bean, clover, apple, plum, peach, pear, maple, oak, and elm. All plants which are a food source for Lepidoptera or Diptera insects and which have agriculture, horticulture, and/or forestry value are plants that are covered by this invention and are referred to as “economically valuable plants”.

Lepidoptera is an order of insects that covers moths and butterflies. Non-limiting examples of Lepidoptera include the following insects. Armyworms and cutworms of the Noctuidae family eat grains and vegetables and include Heliothis zea (Boddie) (also known as corn earworm) and tortricid Cydia pomonella (Linnaeus) (also known as codling moth) which eat orchard crops. Forest defoliators include Choristoneura fumiferana (Clemens) (also known as spruce budworm), C. occidentalis, the geometrid Lambdina fiscellaria lugubrosa (Hulst) (also known as the western hemlock looper), Orgyia pseudotsugata (McDunnough) (also known as Douglas-fir tussock moth), and tent caterpillars of the Lasiocampidae family. Lepidoptera species utilize all parts of plants, including roots, trunk, bark, branches, twigs, leaves, buds, flowers, fruits, seeds, galls and fallen material. Lepidoptera larvae which feed in a concealed manner are wood borers, leaf and bark miners, casebearers, leaf tiers and leaf rollers. Lepidoptera larvae which feed in an exposed manner include Zygaenidae (burnet moths), a large family of day-flying moths.

Diptera insects include flies, gnats, maggots, midges, mosquitoes, keds, and bots. The phytophagous species feed on various parts of plants, dead or alive. The larvae of Tipula oleracea and T. paludosa (also known as leatherjackets which are the larvae of crane-flies or daddy-long-legs) can destroy grass-lands. Ceratitis capitata and Dacus spp. eat fruits. Mayetiola destructor (also known as Hessian-fly) Oscinis spp., and Chlorops spp. eat wheat and other crops. Some leaf miners are in Diptera. Lycoriella spp., Sciara spp., and Bradysia spp. are also known as fungus gnats or mushroom flies and feed on root hairs of plants, including economically valuable plants.

Other insects, not within the Lepidoptera and Diptera orders, also can utilize serine proteases for digestion. Non-limiting examples of such other insects that utilize serine proteases, include Lygus Hesperus, L. lineolaris, rice brown plant hopper (Nilaparvata lugens), and Ostrinia nubilalis.

Because Lepidoptera and Diptera insects, as well as other insects that utilize serine proteases, can cause immense economic harm by feeding on economically valuable plants, it is useful to increase the plants' resistance to these insects. One mechanism for increasing economically valuable plants' resistance to these insects is to have the plants express the serine proteinase inhibitor, BvSTI, only or in combination with other proteinase inhibitors. One can generate transgenic plants containing BvSTI by using the methods discussed herein or using methods known to one of ordinary skill in the art. The expression levels of BvSTI in transgenic plants may be the same or higher than in plants containing BvSTI gene naturally.

Having now generally described this invention, the same will be better understood by reference to certain specific examples and the accompanying drawings, which are included herein only to further illustrate the invention and are not intended to limit the scope of the invention as defined by the claims. The examples and drawings describe at least one, but not all embodiments, of the inventions claimed. Indeed, these inventions may be embodied in many different forms and should not be construed as limited to the embodiments set forth herein; rather, these embodiments are provided so that this disclosure will satisfy applicable legal requirements.

### Example 1

### Generation of B. vulgaris ESTs

Tetanops myopaeformis (sugar beet root maggots) first- and second-instars are collected from fields near St. Thomas, N. Dak. by Larry Campbell (ARS, Fargo, N. Dak.). Fifteen B. vulgaris seedling strains F1016 and F1010 are washed to remove soil and placed on 150 mm×10 mm water/agar (0.8%) plates. The F1010 strain of B. vulgaris is susceptible to sugar beet root maggots whereas F1016 strain demonstrates some resistance. Five first- or second-instar T. myopaeformis are placed on the root of each seedling and allowed to feed for twenty-four or forty-eight hours. The roots and a small amount of hypocotyls tissue are separated from the seedling, rinsed with water to remove the maggots, are frozen in liquid nitrogen, and then stored at −80° C. until RNA isolation.

For the differential screening of cloned sugar beet ESTs, seedlings exposed to chemical and physical wounding are generated. Sugar beet seedlings strain F1016 and strain F1010 are placed in plastic containers with 50 mM NaPO₄ (pH 7.0) supplemented with either 1 mM salicylic acid, 100 μM methyl jasmonate (Thurau et al. 2003. Plant Mol. Biol. 52:643-660), or 1 mM Ethephon (Mazarei et al. 2002. Mol. Plant-Microbe Interact. 15:577-586), which slowly releases ethylene because of a chemical reaction. Roots for wounding treatment are crushed with forceps every centimeter. Control plants are treated identically except the control plants are not wounded nor subjected to chemical treatment. After twenty-four or forty-eight hours, the roots and a small amount of hypocotyls tissue are separated from the seedling, rinsed with water, are frozen in liquid nitrogen, and then stored at −80° C. until RNA isolation.

To prepare RNA, frozen root tissue is ground into a fine powder under liquid nitrogen. Total RNA is isolated by adding 500 μl extraction buffer (0.2 M NaOAc, pH 5.2, 1% SDS, 0.01 M EDTA, 0.5 mg/ml heparin, 0.02 M 2-mercatoethanol) and 500 μl water-saturated re-distilled phenol to approximately 300 mg of frozen plant tissue and then vortexing vigorously. The mixture is then centrifuged and the aqueous phase is removed and placed in new tubes. The organic phase is then re-extracted with 200 μl extraction buffer and centrifuged as before. The aqueous phase from both extractions are combined and extracted with an equal volume of phenol:chloroform:isoamyl alcohol (25:24:1) followed by extraction with chloroform: isoamyl alcohol (24:1). Total RNA is precipitated with 0.33 volumes of 10 M LiCl at −80° C. for one hour. Total RNA is then resuspended in water and quantified spectrophotometrically using Nanodrop 8000 (ThermoFischer Scientific, Waltham, Mass.). RNA quality is assessed using denaturing agarose/formaldehyde gel electrophoresis. Poly A⁺ RNA is purified using DynaBeads (Invitrogen, Carlsbad, Calif.) using provided instructions and quantified spectrophotometrically using Nanodrop 8000 (ThermoFischer Scientific, Waltham, Mass.).

Suppresive subtractive hybridization enriches for genes that are regulated by sugar beet root maggot feeding and possibly are involved in the root's defense response. The suppressive substractive hybridization is conducted using the PCR-Selected cDNA Subtraction Kit (BD Biosciences, Franklin Lakes, N.J.) using provided instructions with 2 μg polyA⁺RNA. Subtraction libraries are obtained (F1010 infested versus uninfested at both 24 hours and 48 hours; F1016 infested versus uninfested at both 24 hours and 48 hours; and F1010 versus F1016 with both infested and uninfested tissue at both 24 hours and 48 hours). The tissue for each subtraction library is a pool of at least three biological replicate experiments. The resulting subtractive libraries are cloned into pCR2.1 TOPO (Invitrogen, Carlsbad, Calif.) vectors and are transformed into TOP10 E. coli (Invitrogen, Carlsbad, Calif.) per the manufacturer's instructions. Transformed bacteria are plated on LB media supplemented with kanamycin (50 μg/ml; LBₖₐₙ), single colonies are placed into 96-well plates containing LBₖₐₙ, grown overnight, supplemented with an equal volume of 60% glycerol and frozen at −80° C.

Forward subtractions (infested cDNA as tester, uninfested cDNA as driver) enriches for up-regulated genes. Reverse subtractions (infested cDNA as driver, uninfested cDNA as tester) enriches for down-regulated genes. Both types of subtractions conducted within each genotype and infestations with first- and second-instar sugar beet root maggots. Second-instars are used in combination with first-instar larvae because the second-instars' larger size manifests as more damage. A pooled sample from 24 hours and 48 hours time points is compared to a pooled sample from uninfested tissue. Approximately 383 clones are picked from each F1010 and F1016 and subjected to differential hybridization. Approximately 288 clones are picked from inter-genotype subtraction in which all F1010 samples (uninfested and infested) are pooled and compared in forward and reverse directions to pooled F1016 samples (uninfested and infested). In total, over one-thousand ESTs are identified.

Differential expression confirmation is conducted as directed by manufacturer's instructions using PCR-Select Differential Screening Kit (Becton Dickinson, Franklin Lakes, N.J.) using the same RNA as is used for the suppressive subtractive hybridization procedure. 100 μl cultures in LBₖₐₙ are grown for 7.5 hours at 37° C., 2 μl culture is used as template for insert amplification. Amplification success is confirmed with gel electrophoresis. 2 μl of PCR reaction are denatured, spotted onto nylon membranes using a 12-channel pipette and neutralized in 0.5 M Tris-HCl (pH 7.0). Membranes are then dried, UV cross-linked and stored under vacuum until hybridization. Forward and reverse subtracted probes are synthesized using a DIG-High Prime DNA Labeling and Detection Starter Kit II (Roche, Basel, Switzerland) per manufacturer's instructions. Probes are quantified per Roche's instructions in order to ensure equal amounts of probe are used in all hybridizations. Pre-hybridizations and hybridizations are conducted at 42° C. for two and sixteen hours, respectively, in DIG Easy Hyb Granules (Roche, Basel, Switzerland) supplemented with a blocking solution as described in the PCR-Select Differential Screening Kit and 0.0623 μg/ml sheared, denatured herring sperm DNA. Blots are washed for two to ten minutes in 2×SSC/0.1% SDS at room temperature and two to fifteen minutes in 0.35×SSC/0.1% SDS at 65° C. Detection of DIG probes are performed as instructed using CSPD Ready-to-Use (DIG-High Prime DNA Labeling and Detection Starter Kit II (Roche, Basel, Switzerland)) except blots are incubated with blocking buffer (supplied in kit) for one hour instead of thirty minutes. Images of the chemiluminescence are gathered using the AlphaImager 3400 (AlphaInnotech, San Leandro, Calif.). Transformed bacteria visually identified as differentially regulated are picked into new 96-well plates, grown overnight in LBₖₐₙ, supplemented with equal volume 60% glycerol, and are used as master plates for sequencing. Individual vectors hybridized only with the expected probe. For example, vectors obtained from the forward subtraction library of F1016 hybridize only to the forward subtracted probe. Approximately 60% of the screened vectors demonstratively hybridized differentially between the forward and reverse probes across all three subtractive procedures.

The vectors in the transformed bacteria which are confirmed to be differentially expressed are sequenced to determine insert size and putative function based on sequence similarity. Sequencing is performed at the DNA Synthesis and Sequencing Facility, Iowa State University (Ames, Iowa). Raw sequences are stripped of contaminating vector sequences and are analyzed by BLASTXZ (Altschul et al. 1997. Nucleic Acids Res. 25:3389-3402) against the GenBank non-redundant database. Batch BLASTN is also conducted against the TIGR B. vularis gene index to identify sugar beet ESTs. Individual ESTs are compared to each other using local BLASTN to identify a unique set of ESTs. Representative individual transformed bacteria of each EST are placed into a new 96-well plate and frozen in 60% glycerol stock and used as the macroarray master plate.

Inserts from the vectors contained in the macroarray master plate set of transformed bacteria are amplified using PCR as described above. Then 5 μl of the PCR reaction, 190 μl water, 210 μl 0.4 M NaOH are mixed at room temperature. Next, 100 μl is spotted onto each of four 96-well dot blotter (Bio-Rad, Hercules, Calif.). After liquid is pulled through the nylon membrane, 200 μl 0.4 M NaOH and 200 μl 2×SSC are sequentially pulled though each well. The membranes are transferred to filter paper presoaked with 0.5 M Tris-HCl (pH 7.0) for four minutes and are air dried. DNA is cross-linked to the membranes with four minute exposure to UV-light from the gel box used for imaging ethidium bromide stained gels. Membranes are stored under vacuum at room temperature until hybridization. Two experiments are conducted, and clones are spotted once in the first experiment or twice in different areas of the nylon membrane in the second experiment.

Most clones contained relatively short inserts with an average insert size of approximately 537 bp over all three subtractions. 121 unique ESTs are identified using the intra-genotype subtractions of the moderately resistant F1016 genotype identified. 42 unique sugar beet root maggot regulated ESTs are identified with the intra-genotype subtractions of the sugar beet root maggot susceptible F1010. Only five ESTs are identified from the inter-genotype subtraction when F1016 cDNA is used as the tester. However, 41 ESTs are identified from the inter-genotype reverse subtraction.

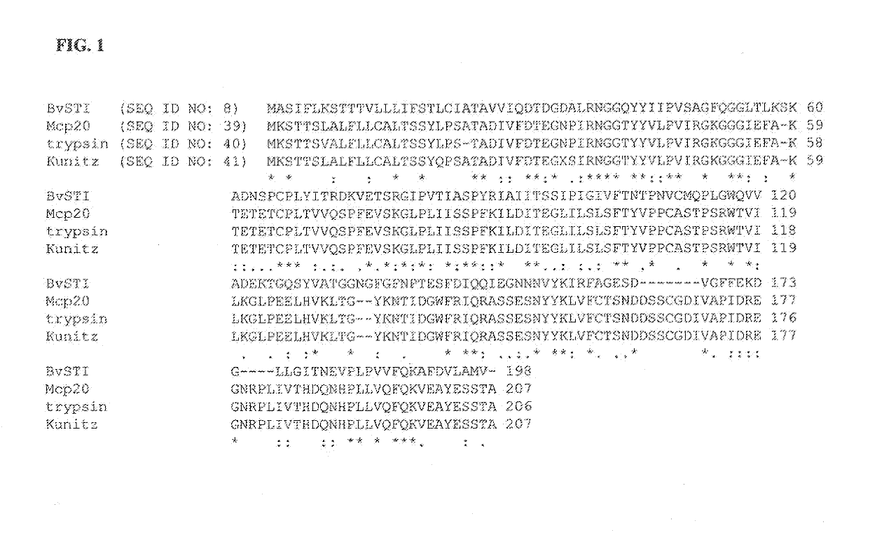

Of the more than 150 ESTs that are up-regulated in response to sugar beet root maggot feeding, an EST is selected for full-length cDNA cloning. This EST, identified as BvSTI, is selected for further analysis because a BLAST analysis of its 227 nucleotides reveals partial homology to a serine proteinase inhibitor (STI) super family conserved domain. 227 nucleotides of the BvSTI EST is submitted to GenBank, submission number DV501688 and made public. The full length coding sequence of BvSTI gene encodes a 198 amino acid sequence that shares approximately 33% homology to three proteinase inhibitors, Mcp20 (GenBank accession number BAB82379.1; Matricaria chamomilla); trypsin inhibitor p20 (GenBank accession number NP_{—}001237952.1; Glycine max), and Kunitz trypsin inhibitor p20-1-like protein precursor (GenBank accession number NP_{—}001237716.1; Glycine max), present in other plants. See FIG. 1.

### Example 2

### Cloning of BvSTI cDNA

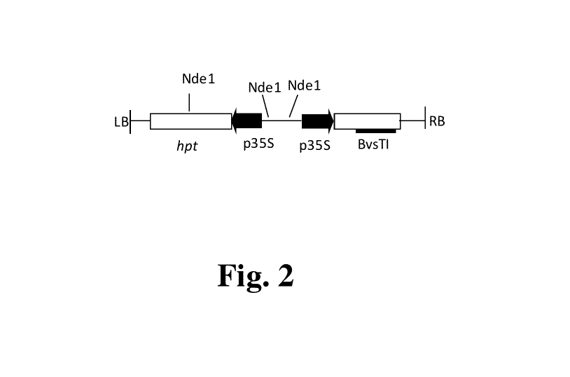

The full length coding sequence of the BvSTI gene is obtained from the BvSTI EST sequence using 5′ and 3′ RACE (BD Biosciences, San Jose, Calif.) and the following primers: 5′ RACE, 5′-CCATTTCTCAGTGCATCGCCGTCTGTGTCT-3′ (SEQ ID NO: 1); and 3′ RACE, 5′-AGACACAGACGGCGATGCACTGAGAAATGG-3′ (SEQ ID NO: 2). The full-length BvSTI gene is then amplified from sugar beet line F1016 (Cambpell et al. 2000. Crop Sci. 40:867-868) by RT-PCR using primers: forward 5′ACCATGGCTTCCATTTTCCTGAAATC 3′ (SEQ ID NO: 3) and reverse 5′GGTCACCTAGACCATCGCTAAAACATCA 3′ (SEQ ID NO: 4) that have NcoI and BstEII restriction enzyme sites, respectively, built in for ease of sub-cloning. Total RNA is prepared using the protocol described above in Example 1. A cDNA of BvSTI is obtained using a Titanium RT-PCR kit (Clontech Laboratories, Inc., Mountain View, Calif.) according to manufacturer's instructions. The full length BvSTI coding sequence is cloned behind the CaMV35S promoter in the pCAMBIA1301 plant transformation vector (CAMBIA, Can berra, Australia) per manufacturer's instructions to yield pBvSTI (see FIG. 2). pCAMBIA1301 carries the hpt marker gene for selection of hygromycin resistant transformed plant cells. The CaMV35S is a constitutive promoter. The full length cDNA (597 bp) sequence of BvSTI is in SEQ ID NO: 7 and the amino acid sequence is in SEQ ID NO: 8.

### Example 3

### BvSTI Expression in Transgenic Nicotiana benthamiana

To confirm the function of the BvSTI proteinase inhibitor in insect resistance, pBvSTI is transfected into transgenic N. benthamiana plants. First, pBvSTI is transferred into A. tumefaciens strain EHA105 per manufacturer's instructions. Next, N. benthamiana leaf disks are excised and are inoculated with Agrobacterium tumefaciens strain EHA105 that carry the pBvSTI transformation vector according to the protocol in Smigocki, et al., 2008 Sugar Tech. 10: 91-98. Putative transformants are selected on Murashige and Skoog media containing B5 vitamins (Murashige and Skoog, 1962. Physiologia Plantarum 15:473-479) and 20 mg hygromycin sulfate/1 (Smigocki et al., 2008; Smigocki et al. 2009b). Regenerated shoots are excised and placed on the same media for rooting prior to transfer to soil. After acclimation, plants are grown in the greenhouse and maintained at 20° C. to 30° C. during the day and 18° C. to 25° C. at night with a day length of 14 to 16 hours. All plants are fertilized monthly with Osmocote (Scott's Miracle-Gro, Marysville, Ohio). T2 progeny homozygous for hygromycin resistance are selected from the T1 progeny of independently derived T0 transgenic plants. The independently derived T2 homozygous progeny exhibit phenotypes that are indistinguishable from the normal, untransformed control plants.

### Example 4

### Confirmation of BvSTI Integration into Transformants' Genome and Presence of BvSTI mRNA in Transformants

To confirm the integration of BvSTI into the T2 N. benthamiana genome, Southern blot analysis of the T2 homozygous lines 11-4, 11-5, 11-6, 11-13 and 12-2 is performed. Genomic DNA is purified using the CTAB (hexadecyltrimethylammonium bromide, Sigma, St. Louis, Mo.) extraction method (Haymes, 1996. Plant Mol. Biol. Rep. 14 (3):280-284). DNA concentration and purity are determined using an ND-8000 Spectrophotometer (NanoDrop Technologies Inc., Wilmington, Del.). Approximately 10 μg of DNA from each plant is digested with NdeI restriction enzyme (New England Biolabs, Inc., Ipswich, Mass.), and is separated by electrophoresis on 1% agarose gels (Sigma Aldrich, St. Louis, Mo.). The DNA is then transferred to a positively charged nylon membrane (Roche, Basel, Switzerland) in 10×SSC (8.76% NaCl and 4.41% sodium citrate, pH 7.0). Membranes are hybridized in DIG Easy Hyb (DIG High Prime DNA Labeling and Detection Starter Kit II, Roche, Basel, Switzerland) with DIG-labeled probes prepared using the PCR DIG Probe Synthesis Kit (Roche, Basel, Switzerland) per manufacturer's instructions. To detect BvSTI, a 0.36 Kb of partial coding region fragment of BvSTI is used as a probe (SEQ ID NO: 36). Detection of DIG probes is carried out as directed by manufacturer's instructions using CSPD Ready-to-Use (DIG-High Prime DNA Labeling and Detection Starter Kit II; Roche, Basel, Switzerland) using forward primer (SEQ ID NO: 37) and reverse primer (SEQ ID NO: 38) and visualized on Lumi-film chemiluminescent detection film (Roche, Basel, Switzerland). T2 homozygous line 11-4 has a faint band; T2 homozygous line 11-6 has a slightly brighter band, T2 homozygous line 11-5 has an even brighter band, and T2 homozygous lines 11-13 and 12-2 have the brightest bands. Each band is positioned above the 5.1 kb marker and the NdeI restricted pBvSTI with a band at approximately 5.1 kb. Thus, the Southern blot analysis confirms that at least a single copy of the BvSTI gene is integrated into the genome of each of the N. benthamiana T2 homozygous lines 11-4, 11-5, 11-6, 11-13 and 12-2.

Next, RT-PCR analysis is used to examine the relative amount of BvSTI mRNA present in each of the N. benthamiana T2 homozygous lines 11-4, 11-5, 11-6, 11-13 and 12-2. To assist with determining the relative amounts of mRNA present, the level of BvSTI mRNA is normalized to the constitutively expressed N. benthamiana actin gene. Total RNA is isolated using RNeasy Plant Mini Kit (Qiagen, Germantown, Md.) per manufacturer's instructions from approximately 100 mg of fresh leaf tissue and treated with RNase-free DNase (Qiagen, Germantown, Md.). Titanium One-Step RT-PCR Kit (Clontech Laboratories Inc., Mountain View, Calif.) is used per manufacturer's instructions to amplify the BvSTI transgene transcripts from about 100 ng of total RNA under the following conditions: 50° C. for 1 hour, 94° C. for 2 minute 40 seconds, followed by 30 cycles of 94° C. for 30 seconds, 60° C. for 40 seconds, 72° C. for 1 minute 30 seconds, ending with the final extension at 72° C. for 5 minutes. BvsTI gene specific primers are used to amplify the 0.6 Kb coding region using forward primer SEQ ID NO: 3, and reverse primer SEQ ID NO: 4 (Smigocki et al., 2008). To normalize the RT-PCR results, transcripts of the constitutively expressed N. benthamiana actin gene are used as loading controls. The following actin primers are used (Forward 5′-GTATTGTKAGCAACTGGGATGA-3′ (SEQ ID NO: 5) and Reverse 5′-AACKYTCAGCCCRATGGTAAT-3′ (SEQ ID NO: 6)) to amplify a 0.54 Kb fragment using the same conditions as described above. The RT-PCR assays are repeated two times with comparable results.

RT-PCR assays reveal high levels of BvSTI mRNA in each of the transformants (11-4, 11-5, 11-6, 11-13 and 12-3) with a large band at approximately 0.6 Kb, and no detectable mRNA in an untransformed N. benthamiana control. The BvSTI mRNA levels are normalized to the constitutively expressed actin mRNA which had a band at approximately 0.54 Kb that was not as large as the band for the BvSTI mRNA. Elevated levels of BvSTI gene transcripts driven by the constitutive CaMV35S promoter are detected in all analyzed T2 homozygous plants.

### Example 5

### Confirmation of BvSTI Production in Transformant Plants

To confirm the presence of the recombinant protein in the T2 transformed lines, a Western blot analysis with BvSTI-specific polyclonal antibodies is performed. First, proteins in the transgenic 11-4, 11-6, 11-13, and 12-2 Nicotiana plants are extracted from leaves previously ground into a fine powder under liquid nitrogen in ice cold 50 mM Tris-HCl pH 7.5, 150 mM NaCl, 10 mM EDTA, 10% sucrose, 10 mM ascorbic acid, 1 mM PMSF, 2 mM DTT in proportion of 10 ml extraction buffer per 1 g of tissue (Chan and De Lumex, 1982. J. Agric. Food Chem. 30:42-46; Wang et al. 2003. Plant Sci. 165:191-203; Smigocki et al. 2008; Smigocki et al. 2009. Plant Cell Tiss. Organ Cult. 97(2):167-174 (hereinafter Smigocki et al. 2009(a))). After centrifugation at 10,000 rpm for 10 minutes, the supernatant (crude extract) is concentrated to about 1 ml using Amicon Ultra 15 (3K) concentrator (Millipore, Billerica, Mass.) by centrifugation at 4° C. The concentrated extract is desalted in 8.5 ml of 62.5 mM Tris-HCl, pH 6.8 two times and is centrifuged until the retentate volume is less than 200 μl. Total proteins are quantified according to Bradford 1976. Anal. Biochem. 72:248-254.

Next, total protein isolated (15 μg or 30 μg) are separated on 12% SDS-PAGE gels in 0.025 M Tris, 0.192 M glycine and 3.5 mM SDS running buffer. In addition, BvSTI peptides, used for the production of anti-BvSTI antibodies, are loaded onto the gel for a positive control. After electrophoresis, gels are equilibrated in cold transfer buffer (0.025 M Tris, 0.192 M glycine, 0.025% SDS) for 1 hour. Separated proteins are subsequently transferred to Immun-Blot PVDF Membranes (0.2 μm, Bio-Rad, Hercules, Calif.) for 1 hour 20 minutes at 70 V (Bio-Rad Mini-Trans-Blot Electrophoretic Transfer cell, Bio-Rad, Hercules, Calif.). Following transfer, membranes are rinsed in deionized water and gently agitated in blocking solution (5% BLOT-QuickBlocker, Chemicon International (now Millipore), Billerica, Mass.) for 1 hour. Membranes are then incubated with rabbit anti-BvSTI antibodies (GenScript Inc., Piscataway, N.J.) produced to a mixture of two most antigenic BvSTI peptides at 1:2000 or 1:5000 (v/v) dilutions in 1×TBS-T (0.137 M NaCl, 0.02 M Tris pH 7.6, 0.1% Tween 20). After 1 hour 30 minutes incubation, membranes are rinsed two times in 1×TBS-T for 10 minutes each, and are incubated for 1 hour in alkaline phosphatase conjugated secondary antibody (AP Conjugated Goat anti-Rabbit IgG, 1:5000 diluted in 1×TBS-T, Chemicon International (now Millipore), Billerica, Mass.). Membranes are washed in 1×TBS-T two times for 15 minutes and then 1 minute in 1×TBS to remove the Tween 20. Alkaline phosphatase is detected using BCIP/NBT (5-bromo-4-chloro-30-indolylphosphate p-toluidine salt and nitro-blue tetrazolium chloride, respectively (Roche, Basel, Switzerland)) in 0.1 M Tris-HCl pH 9.5, 0.1 M NaCl, and 0.05 M MgCl₂ (Savic and Smigocki 2012; Smigocki et al. 2009b). Experiments are repeated two times.

Proteins of approximately 22 to 25 and 30 kDa cross-reacted with the anti-BvSTI antibodies in the transgenic 11-4, 11-6, 11-13, and 12-2 Nicotiana plants. Overall, protein concentrations are low in all of the analyzed transformants, and no cross-reacting 22 to 25 and 30 kDa proteins are detected in the untransformed control. In the positive control lane, BvSTI peptides (5 μg of each peptide) that was used for production of the anti BvSTI-specific antibody and that were loaded 60 minutes after the beginning of electrophoresis are detected. Molecular weight standard proteins that correspond to bands at approximately 30 kDa, 31.2 kDa, and 37.1 kDa are observed.

### Example 6

### BvSTI Proteinase Inhibitor Activity Determination

To determine the level of BvSTI proteinase inhibitor activity, total protein extracts from the transgenic 11-4, 11-6, 11-13, and 12-2 Nicotiana plant leaves are analyzed using an in-gel trypsin inhibitor activity assay. The proteins are extracted from the transgenic plants as described above in Example 5. 15 μg of total protein are separated by SDS-polyacrylamide gel electrophoresis (SDS-PAGE). Gels are incubated with gentle shaking in 25% (v/v) 2-propanol, 10 mM Tris-HCl pH 7.4 for 30 minutes to remove SDS followed by 10 mM Tris-HCl pH 8.0 for another 30 minutes to renature the proteins (Smigocki et al., 2008; 2009a; Cai et al. 2003. Plant Mol. Biol. 51:839-849; Wang et al. 2003; Savic and Smigocki 2012; Smigocki et al. 2008; Smigocki et al. 2009b). Gels are then soaked with 40 μg/ml bovine trypsin (Sigma Aldrich, St. Louis, Mo.) in 50 mM Tris-HCl pH 8.0, 50 mM CaCl₂ for 40 minutes and are transferred to a freshly prepared substrate-dye solution consisting of 2.5 mg/ml N-acetyl-DL-phenylalanine β-naphthyl ester (Sigma Aldrich, St. Louis, Mo.) suspended in dimethylformamide and 0.5 mg/ml tetrazotized O-dianisidine (Sigma Aldrich, St. Louis, Mo.) suspended in 50 mM Tris-HCl pH 8.0 with 50 mM CaCl₂, for 30 minutes at room temperature. Acetic acid (10%) is added to stop the reaction. Clear zones corresponding to proteins with trypsin inhibitory activity are recorded. The assay is repeated two to three times with comparable results.

Multiple clear zones (white bands) corresponding to trypsin inhibitor activity of approximately 30, 28 and 26 kDa are detected in transformants 11-4, 11-5, 11-6, 11-13 and 12-2 that are not observed in the untransformed control plant lane. A unique and distinct clear zone at approximately 30 kDa is detected in all five homozygous BvSTI transformants by the gel trypsin activity assay. In addition to the expected band at approximately 30 kDa BvSTI, two additional zones of activity corresponding to approximately 28 and 26 kDa are clearly visible in the lanes for transformants 11-5 and 11-13. Transformants 11-4 and 11-6 have reduced levels of the active 28 and 26 kDa trypsin inhibitors as compared to transformants 11-5 and 11-13 based on intensity and size of the bands. Transformant 12-2 has the lowest level of the active 30 kDa BvSTI protein with greatly reduced 28 kDa and no detectable 26 kDa activity. The negative control plants lacked any trypsin inhibitory activity at these molecular weights.

While not intending to be held to any particular theory, the low levels of detected 30 kDa BvSTI in the Western blot may result from possible high turnover and/or modification of BvSTI in Nicotiana, despite high transcription of BvSTI by the expression vector and high activity in the gel trypsin activity assay. Interestingly, no cross-reactivity of the BvSTI-specific antibody with the approximate 28 kDa and 26 kDa proteins is observed by Western blots. Not intending to be held to any particular theory, it is possible that these less abundant 28 kDa and 26 kDa proteins represent modified or partially degraded forms of the 30 kDa BvSTI.

### Example 7

### Insect Feeding Resistance (Leaf Feeding)

The five independently derived N. benthamiana transgenic plants, 11-4, 11-5, 11-6, 11-13, and 12-2, which have demonstrably high levels of BvSTI gene expression and detectable hydrolytic trypsin activity (as described above) are used to assess their resistance to five Lepidoptera insects. These insect feeding assays are conducted to study the effect of the sugar beet BvSTI proteinase inhibitor on growth and development of Lepidoptera insects. Newly emerged fall armyworm (Spodoptera frugiperda J. E. Smith), beet armyworm (Spodoptera exigua Hubner), black cutworms (Agrotis ipsilon Hufnagel) and tobacco budworm (Heliothis virescens Fabricius) larvae are purchased from Benzon Research (Carlisle, Pa.) and are reared on the artificial diet provided by Benzon Research. The larval insects are maintained at room temperature for approximately one to approximately three days and are removed from the diet approximately two hours prior to the start of the insect feeding experiments. For leaf assays, a fully expanded leaf from a 4-month old greenhouse grown Nicotiana plant (either a transgenic plant or a normal plant) is placed on water moistened filter paper in a Petri dish and is infested with weighed larva (second instar) for each insect. The Petri dish containing the leaf and insect larva are kept in the dark at room temperature, and larval weights and mortality are recorded daily until pupation. Each experiment is repeated between two to five times with each experiment containing between five and ten separate leaves (replicates) for that particular insect. The leaf assays are conducted with the transformant N. benthamiana plants, 11-4, 11-5, 11-6, 11-13, and 12-2.

Second-instars of the fall armyworm (Spodoptera frugiperda J. E. Smith), a generalist lepidopteran herbivore with a wide host range, are provided with a leaf from one of the five N. benthamiana transgenic plants, 11-4, 11-5, 11-6, 11-13, and 12-2, or a non-transgenic leaf as a negative control. Daily observations are made to determine survival, weight gain and developmental stage of the larvae. Larvae are weighed at the start of the experiment and only those larvae with non-significantly different weights are used in the bioassay. Larvae feeding on leaves from BvSTI transformed plants 11-4, 11-5, 11-6, 11-13 and 12-2 have significantly reduced mean larval weights at three (31 to 43 mg; except line 12-2), six (48 to 95 mg) and eight (74 to 105 mg; except line 12-2) days as compared to the negative control larval weights of 63 mg, 143 mg, and 258 mg, respectively (see Table 1). In percentage terms, the larvae that feed on the transgenic plants weigh approximately 19% to 51%, approximately 34% to 66%, and approximately 59% to 71% less at three, six, and eight days respectively compared to larvae that feed on the negative control plant.

TABLE 1
Fall armyworm larvae weights after feeding on BvSTI transformants
11-4, 11-5, 11-6, 11-13, or 12-2 or a negative control N. benthamiana
(not containing BvSTI gene) at the indicated number of days.

| BvSTI | | | | |
|---|---|---|---|---|
| Transformants | 3 days | 6 days | 8 days | 10 days |
| 11-4 | 31 ± 4.1^{a} (15) | 48 ± 9.2^{a} (11) | 76 ± 15.9^{a} (9) | 131 ± 33.6^{a} (8) |
| 11-5 | 43 ± 6.5^{a} (15) | 70 ± 8.2^{a} (14) | 105 ± 13.0^{a} (13) | 162 ± 23.1^{b} (12) |
| 11-6 | 32 ± 3.1^{a} (13) | 52 ± 6.4^{a} (11) | 74 ± 9.9^{a} (11) | 106 ± 16.5^{a} (10) |
| 11-13 | 39 ± 3.2^{a} (14) | 55 ± 8.4^{a} (13) | 84 ± 9.3^{a} (11) | 112 ± 12.0^{a} (9) |
| 12-2 | 51 ± 7.8^{b} (15) | 95 ± 19.2^{a} (14) | 157 ± 35.5^{b} (12) | 183 ± 38.0^{b} (11) |
| Negative | 63 ± 7.7^{b} (15) | 143 ± 23.9^{b} (13) | 258 ± 42.2^{b} (11) | 234 ± 25.4^{b} (8) |
| Control | | | | |

Values represent mean larval weight ± SE.
Means followed by the same superscript within columns are not significantly different (P < 0.05) by one-way ANOVA test.
Number in parenthesis indicates the number of living larvae out of fifteen that are weighed.

At ten days, larval weights of the negative controls are reduced because some larvae start to pupate, unlike the larvae feeding on the transformants. In general, an approximate one to three day delay in onset of pupation is observed for larvae feeding on the BvSTI transformed leaves. Pupal sizes reflect the overall larval weights at pupation, i.e., smaller and lighter brown in color for the larvae feeding on the transgenic leaves as compared to the larger and darker negative controls. The rate of pupae emergence from the larvae fed transformant plants or the negative control plant is comparable, and all moths have a similar appearance. Experiments are repeated two more times and significantly reduced larval weights are observed at days three, five, six, seven and eight for larvae feeding on the BvSTI transformants. No significant differences in larval mortality rates are noted (see Table 1). At day three, six and eight, larval mortality averages 4%, 16% and 25% for the lavae that feed on the transformant plants as compared to 0%, 13%, and 27% for the lavae that feed on the negative controls, respectively.

Second-instars of the beet armyworm (Spodoptera exigua Hubner) are provided with a leaf from one of the five N. benthamiana transgenic plants, 11-4, 11-5, 11-6, 11-13, and 12-2, or a non-transgenic leaf as a negative control. Daily observations are made to determine survival, weight gain and developmental stage of the larvae. Larvae are weighed at the start of the experiment and only those larvae with non-significantly different weights are used in the bioassay. Larval weights are reduced at five and seven days of feeding on BvSTI transformed plants 11-4, 11-5, 11-6, 11-13 and 12-2 when compared to larval weights on the negative control plant. However, the reduced weights are only significant on larvae feeding on BvSTI transformant 11-4 and 11-5 at five days (87 mg and 88 mg compared to 139 mg for the negative control) (see Table 2). In a repeat experiment, all larval weights are similarly reduced, however, only the larvae feeding on transformants 11-6 and 11-13 have significant reduction in their weights (179 mg and 190 mg, respectively compared to 233 mg for the negative control; data not shown). No significant differences in larval mortality or pupation are noted. A higher incidence of pupae displaying abnormal development (deformed wings and/or smaller size) and/or non-emergence is observed for the beet armyworm larvae that feed on transgenic leaves.

TABLE 2
Beet armyworm larvae weights after feeding on BvSTI
transformants 11-4, 11-5, 11-6, 11-13, or 12-2 or a negative
control N. benthamiana plant (not containing BvSTI
gene) at the indicated number of days.

| BvSTI | | | |
|---|---|---|---|
| Transformants | 0 days | 5 days | 7 days |
| 11-4 | 38 ± 2.0 (8) | 87 ± 13* (7) | 116 ± 27 (6) |
| 11-5 | 38 ± 2.0 (8) | 88 ± 15* (7) | 108 ± 18 (6) |
| 11-6 | 36 ± 2.0 (8) | 109 ± 18 (8) | 183 ± 30 (7) |
| 11-13 | 38 ± 2.0 (8) | 109 ± 9.2 (8) | 160 ± 25 (5) |
| 12-2 | 36 ± 1.0 (8) | 108 ± 13 (8) | 125 ± 23 (7) |
| Negative | 37 ± 1.0 (8) | 139 ± 20 (8) | 168 ± 27 (7) |
| Control | | | |

Values represent mean larval weight ± SE.
*= significant at P < 0.05 as compared to the negative control.
Number in parenthesis indicates the number of living larvae out of eight that are weighed.

Second-instars of the black cutworm (Agrotis ipsilon Hufnagel) larvae are provided with a leaf from one of the five N. benthamiana transgenic plants, 11-4, 11-5, 11-6, 11-13, and 12-2, or a non-transgenic leaf as a negative control. Daily observations are made to determine survival, weight gain and developmental stage of the larvae. Larvae are weighed at the start of the experiment and only those larvae with non-significantly different weights are used in the bioassay. At three, five and seven days after initiation of feeding, average weights of the larvae feeding on all five BvSTI transformant plants are higher than the average weights of the larvae feeding on the negative control leaves (see Table 3). Average weights for the larvae feeding on the transformant plants at three days range from 116 mg to 158 mg and are significantly higher than the average weights of the larvae (63 mg) feeding on the negative control plant, except for larvae feeding on BvSTI transformant 11-6 (116 mg). At five days, larval weights range from 141 mg to 202 mg for the larvae feeding on the transformant plants and 81 mg for the larvae feeding on the negative control plant; the weights of the larvae feeding on transformant plant 12-2 being significantly higher. Similar increases in larval weights are also observed at seven days, averaging approximately 282 mg for the larvae feeding on the transformant plants compared to 197 mg for the larvae feeding on the negative control plants. In repeat experiments, similar increases in larval weights are noted for the larvae feeding on the transgenic plants compared to the larvae feeding on the negative control plants. No differences in larval mortality are observed, and pupal sizes reflect the increased larval weights, as did the emerging moths.

TABLE 3
Black cutworm mean larval weights after feeding on BvSTI
transformants 11-4, 11-5, 11-6, 11-13, or 12-2 or a negative
control N. benthamiana plant (not containing BvSTI
gene) at the indicated number of days.

| BvSTI | | | |
|---|---|---|---|
| Transformants | 3 days | 5 days | 7 days |
| 11-4 | 136 ± 22^{a} (5) | 173 ± 37^{b} (5) | 330 ± 87^{a} (5) |
| 11-5 | 129 ± 24^{a} (5) | 165 ± 37^{b} (5) | 266 ± 75^{a} (5) |
| 11-6 | 116 ± 8.4^{b} (5) | 168 ± 18^{b} (5) | 299 ± 30^{a} (5) |
| 11-13 | 128 ± 20^{a} (5) | 141 ± 31^{b} (4) | 202 ± 59^{a} (4) |
| 12-2 | 158 ± 31^{a} (5) | 202 ± 18^{a} (4) | 315 ± 36^{a} (4) |
| Negative | 63 ± 36^{b} (4) | 81 ± 39^{b} (3) | 197 ± 0 (1)† |
| Control | | | |

Values represent mean larval weight ± SE.
Means followed by the same superscript within columns are not significantly different (P < 0.05) by one-way ANOVA test.
Number in parenthesis indicates the number of living larvae out of 5 that were weighed;
†only 1 larvae weighed, the other 4 pupated.
Data at 7 days are not statistically analyzed.

Second-instars of the tobacco budworm (Heliothis virescens Fabricius) larvae are provided with a leaf from one of the five N. benthamiana transgenic plants, 11-4, 11-5, 11-6, 11-13, and 12-2, or a non-transgenic leaf as a negative control. Daily observations are made to determine survival, weight gain and developmental stage of the larvae. Larvae are weighed at the start of the experiment and only those larvae with non-significantly different weights are used in the bioassay. At five and seven days after initiation of feeding, all larvae feeding on BvSTI transformant plants are heavier than the larvae feeding on the negative control plants (see Table 4). At five days after initiation of feeding, larval weights for larvae feeding on the transformant plants range from 172 mg to 237 mg, with an average weight of 200 mg per larvae. In contrast, the average larval weight for larvae feeding on the negative control plant is 159 mg. At seven days after initiation of feeding, larval weights for the larvae feeding on the transformant plants range from 221 mg to 276 mg, with an average weight of 235 mg per larvae. In contrast, the average larval weight for the larvae feeding on the negative control plant is 191 mg. The increase in larval weights is significant for the larvae fed on transformant 12-2. In two separate repeat experiments, similar increases in larval weights are observed for the larvae feeding on the transgenic plants compared to the larvae feeding on the negative control plant.

TABLE 4
Tobacco budworm mean larval weights after feeding on
BvSTI transformants 11-4, 11-5, 11-6, 11-13, or 12-2 or
a negative control N. benthamiana plant (not containing
BvSTI gene) at the indicated number of days.

| BvSTI | | | |
|---|---|---|---|
| Transformants | 5 days | 7 days | 12 days^{†} |
| 11-4 | 172 ± 14 | 221 ± 16 | 183 ± 21(9) |
| 11-5 | 196 ± 20 | 239 ± 18 | 390 ± 162(7) |
| 11-6 | 198 ± 13 | 217 ± 15 | 206 ± 23 (8) |
| 11-13 | 199 ± 20 | 221 ± 21 | 180 ± 29 (5) |
| 12-2 | 237 ± 17* | 276 ± 15* | 209 ± 6 (5) |
| Negative | 159 ± 15 | 191 ± 16 | 198 ± 16 (9) |
| Control | | | |

Values represent mean larval weight ± SE and number in parenthesis indicates the number of living larvae out of 10.
*= significant at P < 0.05 as compared to the negative control within the column.
^{†}= larvae started to pupate at nine days.

Despite the higher weights for larvae feeding on the transgenic plants, the larval mortality rates between the larvae feeding on the transgenic plants and the negative control plants differ. Larvae that fed on transgenic plants 11-5, 11-6 and 11-13 have a mortality rate three, two, and five times, respectively, the mortality rate observed for the larvae fed the negative control plants, i.e., one out of ten larvae died. Emerging moths (from larvae that fed on the transformed plants) display varying degrees of developmental abnormalities, including wing development and aborted emergence.

While not wanting to be held to any particular theory, the increase in size for the black cutworm and tobacco budworm larvae may result from a sub-lethal concentration of BvSTI which induces a persistent hunger in the larvae and thus compensatory feeding. Such a theory was proposed for an experiment using Heliothis obsolete and Liriomyza trifolii larvae with increased feeding and faster larval growth (Abdeen et al. 2005. Plant Mol. Biol. 57:189-202). Others have observed increased larval weights feeding on proteinase inhibitor transformed plants. See Cloutier et al. 1999. Arch. of Insect Biochem. Physiol. 40, 69-79; Cloutier et al. 2000. Arch. Insect Biochem. Physiol. 44, 69-81; and Lecardonnel et al. 1999. Plant Sci. 140, 71-79.

### Example 8

### Insect Feeding Resistance (Whole Plant Feeding)

Third instar tobacco hornworm (Manduca sexta Linnaeus) are used in a whole plant assay. For this assay, a single transgenic N. benthamiana plant (either 11-4, 11-6, or 11-13) or a non-transgenic N. benthamiana plant as a negative control is placed in a screened cage and is infested with a single third instar tobacco hornworm. The tobacco hornworm larvae are obtained from Lynda Liska (U.S.D.A., Agricultural Research Service, Beltsville, Md.). Larval weights are recorded daily until pupation. The assays are carried out in replicates of three to five plants for each transformant, and the assays are repeated five times. A non-transformed N. benthamiana plant is used as a negative control. At four, six and ten days of infesting the tobacco plant with the tobacco hornworm larvae, all larvae that fed on the BvSTI transformants 11-4, 11-6 and 11-13 have significant lower weights than the tobacco hornworm larvae that fed on the negative control plant, except for transformant 11-6 at day four and ten (see Table 5). At day six, average larval weights range from 1.5 g to 1.9 g for the larvae feeding on the transformant plants compared to 3.7 g for the larvae feeding on the negative control plants. In repeat experiments, the average weights of larvae feeding on transformant plant 11-6 (3.1 g) are significantly reduced compared to the average weight of larvae feeding on the negative control plant (5.1 g) at seven days. No differences in larval mortality are noted, and pupal sizes reflect the larval weights. Varying degrees of abnormal wing development and smaller body sizes that correlate with the reduced larval weights occur on the emerged moths that fed on the BvSTI transformants plants.

TABLE 5
Tobacco hornworm mean larval weights after feeding on BvSTI transformants
11-4, 11-6, or 11-13 or a negative control N. benthamiana plant
(not containing BvSTI gene) at the indicated number of days.

| BvSTI | | | | |
|---|---|---|---|---|
| Transformants | 0 days | 4 days | 6 days | 10 days |
| 11-4 | 0.3 ± .02^{b} (5) | 1.0 ± 0.1^{a} (5) | 1.9 ± 0.3^{a} (5) | 5.0 ± 0.8^{a} (5) |
| 11-6 | 0.3 ± .01^{b} (5) | 1.1 ± 0.2^{b} (5) | 1.9 ± 0.3^{a} (5) | 6.1 ± 0.9^{b} (5) |
| 11-13 | 0.3 ± .01^{b} (5) | 0.8 ± 0.1^{a} (5) | 1.5 ± 0.2^{a} (5) | 4.5 ± 1.0^{a} (5) |
| Negative | 0.3 ± .01^{b} (5) | 1.5 ± 0.2^{b} (4) | 3.7 ± 0.5^{b} (4) | 8.1 ± 0.6^{b} (4) |
| Control | | | | |

Values represent mean ± SE.
Means followed by the same superscript within a column are not significantly different (P < 0.05) by one-way ANOVA test.
Number in parenthesis indicates the number of living larvae out of 5 that are weighed.

All statistical analysis is performed by one-way Analysis of Variance (ANOVA) using Analyse-it software (Analyze-it Software, Ltd., Leeds, United Kingdom). Results are expressed as mean±standard error (S.E.) for the number of replicates in each treatment. The acceptance level of statistical significance was P<0.05.

Fall armyworm, beet armyworm, tobacco hornworm, tobacco budworm and black cutworm cause significant yield losses in hundreds of economically valuable crops and all, with the exception of tobacco hornworm and budworm, infest sugar beet. No experiments are conducted with the sugar beet root maggot because its host range is limited and does not include tobacco.

It is expected that any variation in weight, either decrease or increase, caused by feeding on BvSTI transgenic economically valuable plants will alter the normal life cycle of the insect, thus changing the insect's dynamics and timing of the interaction with the transgenic economically valuable plant; a desirable strategy for enhancing insect tolerance. Because BvSTI transgenic tobacco plants induce some developmental abnormalities of the pupae and the emerging moths, BvSTI transgenic tobacco plants have a negative effect on the insect's life cycle, a strategy for successful control. Because sugar beet is generally grown in geographically limited areas, Lepidoptera, Diptera, and other insects utilizing serine proteases in digestion are less likely to have developed digestive protease resistant to BvSTI, the sugar beet serine proteinase inhibitor, thus making BvSTI a potentially valuable additional tool to protect economically valuable plants.

### Example 9

### Cloning of BvSTI Promoter

A 794 bp promoter for BvSTI is also obtained; see SEQ ID NO: 9. To clone the promoter, a PCR-based strategy is employed using GenomeWalker™ Universal Kit (Clontech, Mountain View, Calif.). Genomic DNA from B. vulgaris strain F1016 is obtained as described above in Example 4. Next, aliquots of genomic DNA are separately digested with the restriction enzymes DraI, EcoRV, PvuII and StuI, and each batch of digested DNA is subsequently ligated to the GenomeWalker Adaptor sequences per manufacturer's instructions. DNA is subjected to PCR per manufacturer's instructions with adaptor specific forward primer (provided in kit) and using a reverse primer containing a nested BvSTI gene specific sequence: reverse: 5′-GATTTCAGGAAAATGGAAGCCAT-3′ (SEQ ID NO: 10). PCR conditions are five cycles at 94° C. for twenty-five seconds followed by 72° C. for three minutes; then followed by twenty cycles at 94° C. for twenty-five seconds and 67° C. for three minutes; and one final cycle at 67° C. for seven minutes. The PCR generated DNA fragment is sequenced to obtain the DNA sequence of the promoter for BvSTI from B. vulgaris strain F1016.

### Example 10

### BvSTI Promoter is an Inducible Promoter

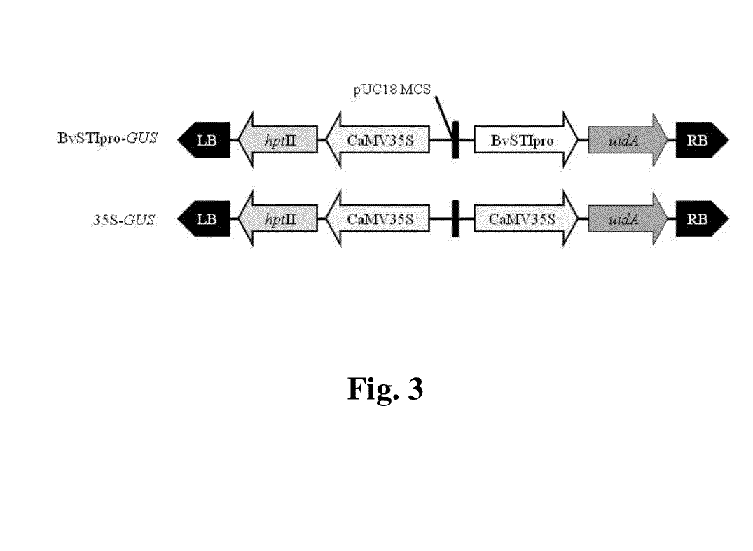

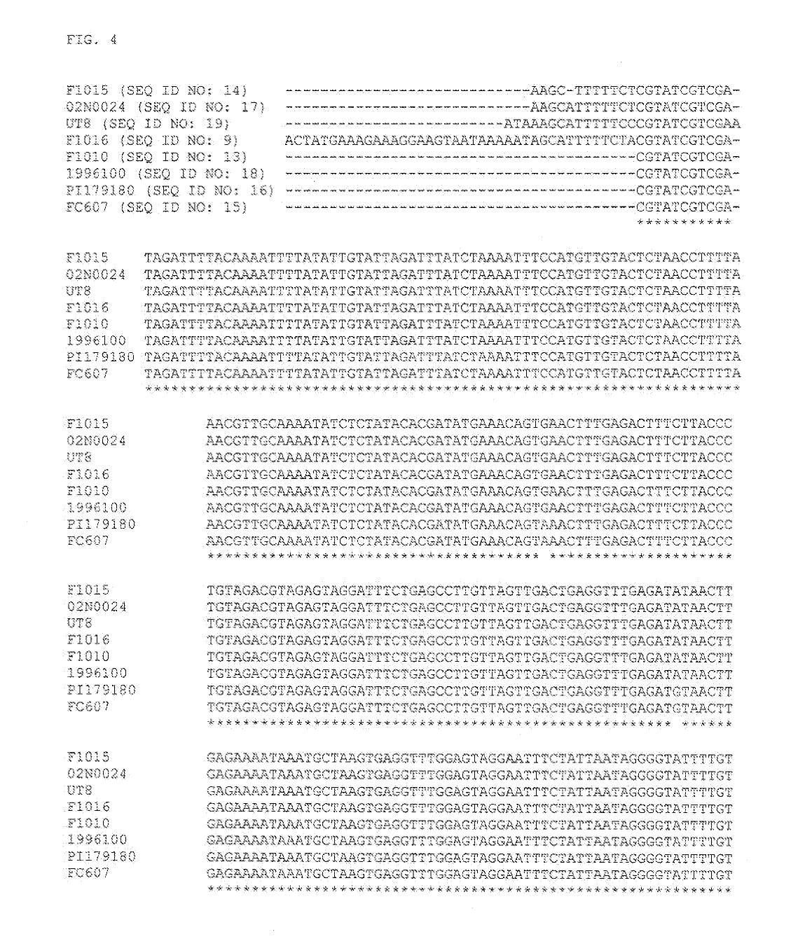

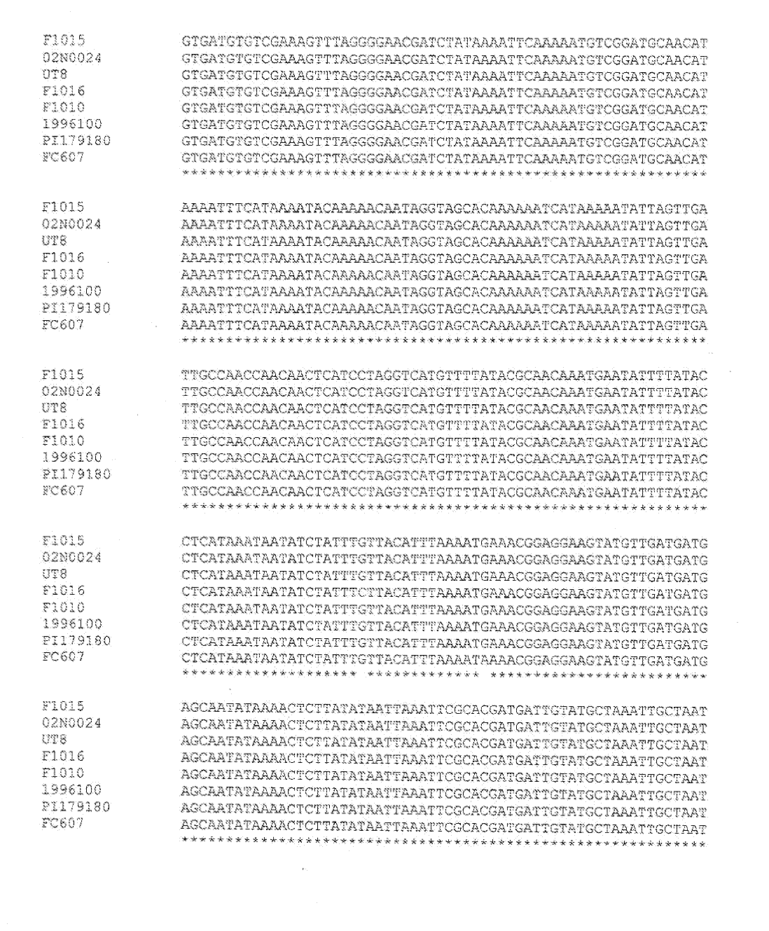

The BvSTI promoter is amplified from F1016 genomic DNA (obtained as described above in Example 4) by PCR using TaKaRa Ex Taq PCR according to manufacturer's instructions (Clontech Laboratories Inc., Mountain View, Calif.) with the following primers: forward 5′-AAGCTTACTATGAAAGAAAGGAAGTAATAA-3′ (SEQ ID NO: 11) containing a HindIII restriction enzyme site built in for ease of sub-cloning into pCAMBIA1301 plant transformation vector and reverse 5′-CCATGGTGTTTTTGTTTGGTGTG-3′ (SEQ ID NO: 12) containing NcoI restriction enzyme site built in for ease of sub-cloning into pCAMBIA1301. BvSTI promoter sequence is cloned upstream of the uidA gene in the pCAMBIA1301 plant transformation vector (CAMBIA, Can berra, Australia) (pBvSTIpro-GUS). pCAMBIA1301 vector carries the htp marker gene for selection of hygromycin resistant transformed plant cells. A pCAMBIA vector with the uidA gene fused to the constitutively expressed Cauliflower Mosaic Virus 35S promoter (CaMV 35S), generating p35S-GUS, is used as a positive control for the transformation process and the activity of uidA gene. See FIG. 3. The uidA gene encodes β-glucoronidase (a.k.a. GUS) which is used as a marker for promoter activity. GUS cleaves 4-methylumbelliferyl-β-D-glucuronide resulting in a blue product that stains the plant tissues blue and is clearly visible by the naked eye.

A. tumefaciens EHA 105 strain harboring either pBvSTIpro-GUS or p35S-GUS are used as inocula for tobacco (N. benthamiana Domin) plants transformation. Prior to co-cultivation, bacteria are grown for two days at 28° C. in YEB liquid medium (Van Larebeke et al. 1977) supplemented with kanamycin and ampicillin in concentrations of 50 mg/l and 100 mg/l, respectively. Bacteria are harvested by centrifugation at 4000×g for ten minutes and resuspended in 30 ml liquid MS (Murashige and Skoog 1962).

Tobacco leaf explants (1 cm²) are cut from fully expanded leaves of greenhouse-grown plants and are surface-sterilized in 70% ethanol and 10% commercial bleach solution, then are washed five times with sterile water. Explants are then placed in the A. tumefaciens bacterial suspension for ten minutes, are blotted dry on sterile filter paper and are placed on nutrition medium containing MS salts, B5 vitamins (Gamborg et al. 1965. In vitro 12(7), 473-478), 3% sucrose and 0.7% agar. After two days of co-cultivation in the dark at 25° C., explants are washed with sterile solutions of cefotaxime and carbenicillin (500 mg/l each) and are placed on agar solidified callus-induction medium (CIM: MS salts, B5 vitamins, 6-benzylaminopurine (BAP) 2 mg/l, 200 mg/l cefotaxime and 500 mg/l carbenicilline). Shoots which regenerate from derived calli are excised and are cultured on ½ B5 selection medium (SM) containing BAP 0.5 mg/l and hygromycin 20 mg/l for proliferation of transformed tobacco lines. Nicely developed 1-2 cm tall shoots with normal phenotype are transferred to rooting medium (RM: ½ B5 medium with no hormones, supplemented with hygromycin 20 mg/l). After few weeks growing in vitro, putatively transformed tobacco plants are acclimated and transferred to greenhouse where they are maintained under controlled environmental conditions (25±5° C. during the day and 22±3° C. over night, with day length of 15±1 h).

Untransformed N. benthamiana plants are included in all experiments as negative controls. To confirm that the transformed N. benthamiana plants contain pBvSTIpro-GUS or p35S-GUS, PCR analysis of genomic DNA obtained from the N. benthamiana plants is performed using TaKaRa Ex Taq PCR according to manufacturer's instructions (Clontech Laboratories Inc., Mountain View, Calif.). Genomic DNA is obtained as described above in Example 4. T2 progeny of the N. benthamiana plants that are demonstrated to be transformed (PCR positive) with either pBvSTIpro-GUS or p35S-GUS are self-fertilized.

The seeds harvested from self-fertilized T1 plants are imbibed overnight in 1000 ppm gibberellic acid (GA₃). After removing the GA₃ solution, the seeds are surface sterilized in 70% ethanol and 10% commercial bleach solution containing 4% sodium hypochlorite for eight minutes. Seeds are then rinsed with sterile water and are germinated on hormone-free 0.6% agar medium supplemented with hygromycin in concentration 40 mg/l in dark. After five days, the plates with germinated seeds are moved to sixteen hours light/eight hours dark conditions. Tobacco seedlings with normal growth are counted as hygromycin resistant and, based on the number of resistant and susceptible plants, the expected segregation ratio for each T2 line is tested using the chi-square (χ²) test (Greenwood and Nikulin 1996. A Guide to Chi-squared Testing, Wiley, NY). All seeds from the greenhouse-grown transformed T1 plants which are tested for hygromycin resistance are resistant to hygromycin. Using the chi-square test, it is believed that a single locus insertion of the hptII gene occurred for all tested T1 plants.

To determine if the BvSTI promoter induces transcription and translation of the uidA gene in leaves of T2 transformed plants in response to insect wounding, fall armyworm larvae are provided leaves from the T2 plants. Larvae that are approximately in the late second instar are placed on up to five leaves from pBvSTIpro-GUS or p35S-GUS transformed plants or negative control plants. The leaves are obtained from plants approximately fourteen weeks old. The larvae and leaves are placed in Petri dishes on wet filter paper. Feeding occur for zero, six, twenty-four, forty-eight or seventy-two hours. At the indicated time points, wounded leaves are collected and dipped into buffer containing 4-methylumbelliferyl-β-D-glucuronide for staining. Each pBvSTIpro-GUS transformant has blue staining localized to the leaf tissue surrounding the site of injury within six hours after feeding. Undamaged areas of leaves lack staining. In contrast, each p35S-GUS transformant has blue staining throughout the leaf, even if the leaf was not wounded. The negative control plants lack blue staining.

To determine if the BvSTI promoter induces transcription and translation of the uidA gene in roots of T2 transformed plants in response to mechanical wounding, roots of pBvSTIpro-GUS or p35S-GUS transformed plants or negative control plants are gently washed in water to remove the soil and then wounded by pinching with forceps at approximately 5 mm intervals over the entire root length. The root of each pBvSTIpro-GUS transformant has blue stain at the site of the mechanical wounding. The root for each p35S-GUS transformant has blue stain throughout the length of the root. The roots of the negative control plant lack blue stain.

These experiments demonstrate that the BvSTI promoter is induced upon wounding of leaves and roots. Using such inducible promoters may be useful in transgenic plants to avoid having BvSTI produced and present throughout the plant.

### Example 11

### BvSTI Promoters and Genes from Other B. vulgaris Strains

In addition to the BvSTI promoter and gene sequence obtained from B. vulgaris strain F1016, the promoter sequences from B. vulgaris strains F1010 (SEQ ID NO: 13), F1015 (SEQ ID NO: 14), FC607 (SEQ ID NO: 15), 02N0024 (SEQ ID NO: 17), 1996100 (SEQ ID NO: 18), and UT8 (SEQ ID NO: 19) are obtained. In addition, the promoter sequence of red beet PI179180 (SEQ ID NO: 16) is also obtained. The DNA and amino acid sequences for BvSTI from B. vulgaris strains, F1010, F1015, FC607, 02N0024, 1996100, and UT8, and red beet strain PI179180 are also obtained. To clone the promoter-genes, genomic DNA is obtained using the methods described in Example 4 above. A PCR-based strategy as described above is employed using the following primer pairs: forward 5′-AAGCTTACTATGAAAGAAAGGAAGTAATAA-3′ (SEQ ID NO: 11, promoter specific) containing a HindIII restriction enzyme site built in for ease of sub-cloning into pCAMBIA1301 plant transformation vector and reverse 5′-GGTCACCTAGACCATCGCTAAAACATCA-3′ (SEQ ID NO: 4, BvSTI gene specific) containing BsTEII restriction enzyme site built in for ease of sub-cloning into pCAMBIA1301. The second PCR uses the following primer pairs that are nested: forward 5′-ATAAAATTCAAAAATGTCGGATG-3′ (SEQ ID NO: 20, primer specific) and reverse 5′-GAGAAATGGTGGACAATACTACA-3′ (SEQ ID NO: 21, BvSTI gene specific). PCR conditions are one cycle 94° C. for two minutes followed by 30 cycles of 94° C. for forty-five seconds, 50° C. for forty-five seconds, and 72° C. for two minutes; with the final extension at 72° C. for seven minutes. Each PCR generated DNA fragment is sequenced to obtain the DNA sequence of the promoter-gene for each sugar beet line. An alignment of the promoter sequences is in FIG. 4. Because of the high degree of homology amongst the promoters listed herein, any of the listed promoters may be used as an inducible promoter, being activated upon wounding of leaves or roots by insect feeding or other injuries.

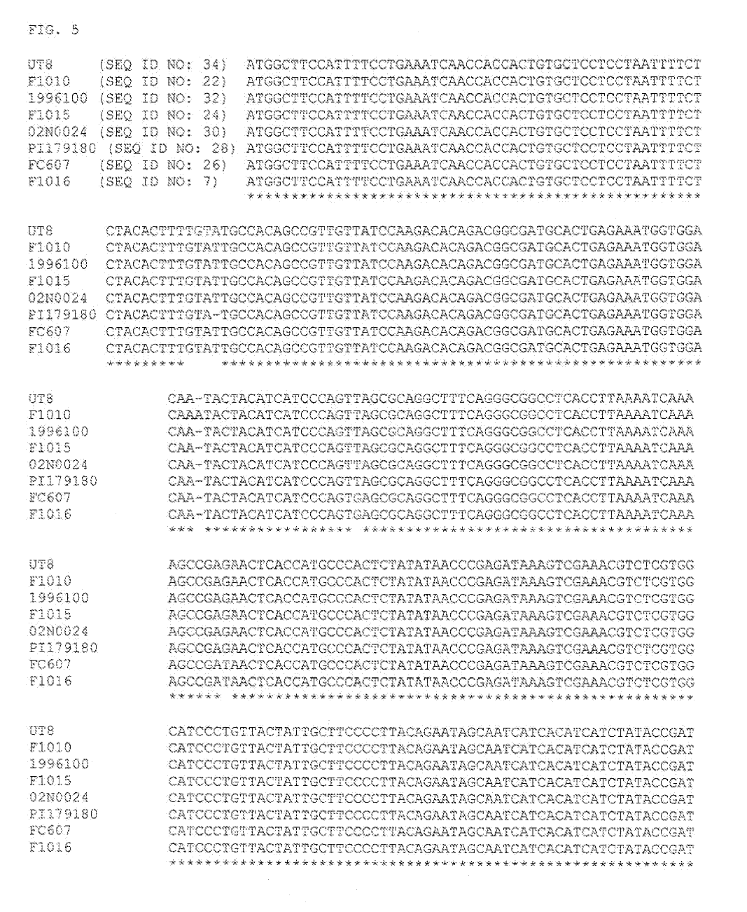

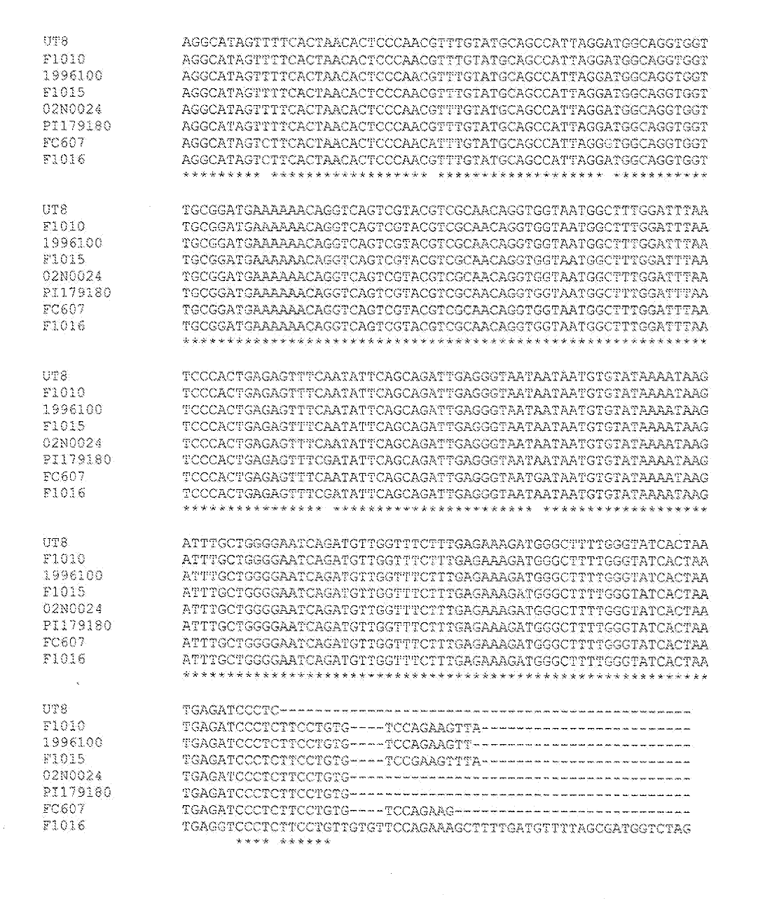

The sequence identification numbers for the DNA sequence and amino acid sequences of BvSTI and BvSTI obtained from these strains are listed in Table 6. An alignment of the cDNA sequences of these strains is in FIG. 5.

TABLE 6

| B. vulgaris | DNA sequence | Amino acid sequence |
|---|---|---|
| strain | identification # | identification # |
| F1010 | SEQ ID NO: 22 | SEQ ID NO: 23 |
| F1015 | SEQ ID NO: 24 | SEQ ID NO: 25 |
| FC607 | SEQ ID NO: 26 | SEQ ID NO: 27 |
| PI179180 (red beet) | SEQ ID NO: 28 | SEQ ID NO: 29 |
| 02N0024 | SEQ ID NO: 30 | SEQ ID NO: 31 |
| 1996100 | SEQ ID NO: 32 | SEQ ID NO: 33 |
| UT8 | SEQ ID NO: 34 | SEQ ID NO: 35 |

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation. All documents cited herein are incorporated by reference.

1. An isolated polynucleotide encoding a serine proteinase inhibitor comprising a polynucleotide having a sequence selected from the group consisting of
SEQ ID NO: 7, SEQ ID NO: 22, SEQ ID NO: 24, SEQ ID NO: 26, SEQ ID NO: 28, SEQ ID NO: 30, SEQ ID NO: 32, or SEQ ID NO: 34;
the full length complements of SEQ ID NO: 7, SEQ ID NO: 22, SEQ ID NO: 24, SEQ ID NO: 26, SEQ ID NO: 28, SEQ ID NO: 30, SEQ ID NO: 32, or SEQ ID NO: 34;
at least 95% identical to SEQ ID NO: 7, SEQ ID NO: 22, SEQ ID NO: 24, SEQ ID NO: 26, SEQ ID NO: 28, SEQ ID NO: 30, SEQ ID NO: 32, or SEQ ID NO: 34;
at least 90% identical to SEQ ID NO: 7, SEQ ID NO: 22, SEQ ID NO: 24, SEQ ID NO: 26, SEQ ID NO: 28, SEQ ID NO: 30, SEQ ID NO: 32, or SEQ ID NO: 34; and
at least 85% identical to SEQ ID NO: 7, SEQ ID NO: 22, SEQ ID NO: 24, SEQ ID NO: 26, SEQ ID NO: 28, SEQ ID NO: 30, SEQ ID NO: 32, or SEQ ID NO: 34.

2. An expression vector comprising said polynucleotide of claim 1.

3. The expression vector of claim 2 wherein said polynucleotide of claim 1 is under control of an inducible promoter or constitutive promoter.

4. The expression vector of claim 3 wherein the inducible promoter is selected from the group consisting of SEQ ID NO: 13, SEQ ID NO: 14, SEQ ID NO: 15, SEQ ID NO: 16, SEQ ID NO: 17, SEQ ID NO: 18, and SEQ ID NO: 19.

5. A transgenic plant cell comprising the expression vector of claim 2, wherein said transgenic plant cell is a cell from an economically valuable plant.

6. The transgenic plant cell of claim 5 wherein said economically valuable plant is a monocot.

7. The transgenic plant cell of claim 5 wherein said economically valuable plant is a dicot.

8. A transgenic plant seed comprising said expression vector of claim 2, wherein said transgenic plant seed is a seed from an economically valuable plant.

9. The transgenic plant seed of claim 8 wherein said economically valuable plant is a monocot.

10. The transgenic plant seed of claim 8 wherein said economically valuable plant is a dicot.

11. A transgenic plant cell comprising said expression vector of claim 4, wherein said transgenic plant cell is a cell from an economically valuable plant.

12. The transgenic plant cell of claim 11 wherein said economically valuable plant is a monocot.

13. The plant cell of claim 11 wherein said economically valuable plant is a dicot.

14. A transgenic plant seed comprising said expression vector of claim 4, wherein said transgenic plant seed is a seed from an economically valuable plant.

15. The transgenic plant seed of claim 14 wherein said economically valuable plant is a monocot.

16. The transgenic plant seed of claim 14 wherein said economically valuable plant is a dicot.

17. A transgenic plant comprising said expression vector of claim 2, wherein said transgenic plant is an economically valuable plant.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A polypeptide comprising the amino acid sequence selected from the group consisting of
SEQ ID NO: 8, SEQ ID NO: 23, SEQ ID NO: 25, SEQ ID NO: 27, SEQ ID NO: 29, SEQ ID NO: 31, SEQ ID NO: 33, or SEQ ID NO: 35;
at least 95% identity to SEQ ID NO: 8, SEQ ID NO: 23, SEQ ID NO: 25, SEQ ID NO: 27, SEQ ID NO: 29, SEQ ID NO: 31, SEQ ID NO: 33, or SEQ ID NO: 35;
at least 90% identity to SEQ ID NO: 8, SEQ ID NO: 23, SEQ ID NO: 25, SEQ ID NO: 27, SEQ ID NO: 29, SEQ ID NO: 31, SEQ ID NO: 33, or SEQ ID NO: 35; and
at least 85% identical to SEQ ID NO: 8, SEQ ID NO: 23, SEQ ID NO: 25, SEQ ID NO: 27, SEQ ID NO: 29, SEQ ID NO: 31, SEQ ID NO: 33, or SEQ ID NO: 35.

36. A transgenic plant comprising an economically valuable plant having an elevated quantity of said polypeptide of claim 35 compared to a wild-type plant.

37. (canceled)

38. (canceled)

39. (canceled)

40. A polynucleotide comprising a promoter having a sequence selected from the group consisting of
SEQ ID NO: 13, SEQ ID NO: 14, SEQ ID NO: 15, SEQ ID NO: 16, SEQ ID NO: 17, SEQ ID NO: 18, or SEQ ID NO: 19; and
at least 95% identity to SEQ ID NO: 13, SEQ ID NO: 14, SEQ ID NO: 15, SEQ ID NO: 16, SEQ ID NO: 17, SEQ ID NO: 18, or SEQ ID NO: 19.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)


SEQUENCE LISTING (38 sequences)

SEQ ID NO: 1 - DNA, 30 bp, Artificial
  Synthesized
ccatttctcagtgcatcgccgtctgtgtct

SEQ ID NO: 2 - DNA, 30 bp, Artificial
  Synthesized
agacacagacggcgatgcactgagaaatgg

SEQ ID NO: 3 - DNA, 26 bp, Artificial
  Synthesized
accatggcttccattttcctgaaatc

SEQ ID NO: 4 - DNA, 28 bp, Artificial
  Synthesized
ggtcacctagaccatcgctaaaacatca

SEQ ID NO: 5 - DNA, 22 bp, Artificial
  Synthesized
gtattgtkagcaactgggatga

SEQ ID NO: 6 - DNA, 21 bp, Artificial
  Synthesized
aackytcagcccratggtaat

SEQ ID NO: 7 - DNA, 597 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagtgagcgcaggctttcagggcggcctcaccttaaaatcaaaagccgataactcaccatgcccactctatataacccgagataaagtcgaaacgtctcgtggcatccctgttactattgcttccccttacagaatagcaatcatcacatcatctataccgataggcatagtcttcactaacactcccaacgtttgtatgcagccattaggatggcaggtggttgcggatgaaaaaacaggtcagtcgtacgtcgcaacaggtggtaatggctttggatttaatcccactgagagtttcgatattcagcagattgagggtaataataatgtgtataaaataagatttgctggggaatcagatgttggtttctttgagaaagatgggcttttgggtatcactaatgaggtccctcttcctgttgtgttccagaaagcttttgatgttttagcgatggtctag

SEQ ID NO: 8 - protein, 198 aa, Beta vulgaris
MASIFLKSTTTVLLLIFSTLCIATAVVIQDTDGDALRNGGQYYIIPVSAGFQGGLTLKSKADNSPCPLYITRDKVETSRGIPVTIASPYRIAIITSSIPIGIVFTNTPNVCMQPLGWQVVADEKTGQSYVATGGNGFGFNPTESFDIQQIEGNNNVYKIRFAGESDVGFFEKDGLLGITNEVPLPVVFQKAFDVLAMV

SEQ ID NO: 9 - DNA, 794 bp, Beta vulgaris
actatgaaagaaaggaagtaataaaaatagcatttttctacgtatcgtcgatagattttacaaaattttatattgtattagatttatctaaaatttccatgttgtactctaaccttttaaacgttgcaaaatatctctatacacgatatgaaacagtgaactttgagactttcttaccctgtagacgtagagtaggatttctgagccttgttagttgactgaggtttgagatataacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttcttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttaaaaccagcatacaaaccttctataaatactagcattcttcagctactaactctcatccccaacatccctcaaacaactatcagtaacatacacaccaaacaaaaacaac

SEQ ID NO: 10 - DNA, 23 bp, Beta vulgaris
gatttcaggaaaatggaagccat

SEQ ID NO: 11 - DNA, 30 bp, Artificial
  Synthesized
aagcttactatgaaagaaaggaagtaataa

SEQ ID NO: 12 - DNA, 23 bp, Artificial
  Synthesized
ccatggtgtttttgtttggtgtg

SEQ ID NO: 13 - DNA, 754 bp, Beta vulgaris
cgtatcgtcgatagattttacaaaattttatattgtattagatttatctaaaatttccatgttgtactctaaccttttaaacgttgcaaaatatctctatacacgatatgaaacagtgaactttgagactttcttaccctgtagacgtagagtaggatttctgagccttgttagttgactgaggtttgagatataacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttaaaaccagcatacaaaccttctataaatactagcattcttcagctactaactctcatccccaacatccctcaaacaactatcagtaacatacacaccaaacaaaaacaac

SEQ ID NO: 14 - DNA, 739 bp, Beta vulgaris
aagctttttctcgtatcgtcgatagattttacaaaattttatattgtattagatttatctaaaatttccatgttgtactctaaccttttaaacgttgcaaaatatctctatacacgatatgaaacagtgaactttgagactttcttaccctgtagacgtagagtaggatttctgagccttgttagttgactgaggtttgagatataacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttaaaaccagcatacaaaccttctataaatactagcattcttcagctactaactctcatccccaacatccctcaaacaactatcag

SEQ ID NO: 15 - DNA, 754 bp, Beta vulgaris
cgtatcgtcgatagattttacaaaattttatattgtattagatttatctaaaatttccatgttgtactctaaccttttaaacgttgcaaaatatctctatacacgatatgaaacagtaaactttgagactttcttaccctgtagacgtagagtaggatttctgagccttgttagttgactgaggtttgagatgtaacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaataaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttcaaaccagcatacaatccttctataaatactagcattcttcatctactaactctcatccccaacatccctcaaacaactatcatcagtaacatacacagcaaacaaaaac

SEQ ID NO: 16 - DNA, 754 bp, Beta vulgaris
cgtatcgtcgatagattttacaaaattttatattgtattagatttatctaaaatttccatgttgtactctaaccttttaaacgttgcaaaatatctctatacacgatatgaaacagtaaactttgagactttcttaccctgtagacgtagagtaggatttctgagccttgttagttgactgaggtttgagatgtaacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttcaaaccagcatacaaaccttctataaatactagcattcttcagctactaactctcatccccaacatccctcaaacaactatcagtaacatacacaccaaacaaaaacaac

SEQ ID NO: 17 - DNA, 766 bp, Beta vulgaris
aagcatttttctcgtatcgtcgatagattttacaaaattttatattgtattagatttatctaaaatttccatgttgtactctaaccttttaaacgttgcaaaatatctctatacacgatatgaaacagtgaactttgagactttcttaccctgtagacgtagagtaggatttctgagccttgttagttgactgaggtttgagatataacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttaaaaccagcatacaaaccttctataaatactagcattcttcagctactaactctcatccccaacatccctcaaacaactatcagtaacatacacaccaaacaaaaacaac

SEQ ID NO: 18 - DNA, 754 bp, Beta vulgaris; identical to SEQ ID NO: 13

SEQ ID NO: 19 - DNA, 770 bp, Beta vulgaris
ataaagcatttttcccgtatcgtcgaatagattttacaaaattttatattgtattagatttatctaaaatttccatgttgtactctaaccttttaaacgttgcaaaatatctctatacacgatatgaaacagtgaactttgagactttcttaccctgtagacgtagagtaggatttctgagccttgttagttgactgaggtttgagatataacttgagaaaataaatgctaagtgaggtttggagtaggaatttctattaataggggtattttgtgtgatgtgtcgaaagtttaggggaacgatctataaaattcaaaaatgtcggatgcaacataaaatttcataaaatacaaaaacaataggtagcacaaaaaatcataaaaatattagttgattgccaaccaacaactcatcctaggtcatgttttatacgcaacaaatgaatattttatacctcataaataatatctatttgttacatttaaaatgaaacggaggaagtatgttgatgatgagcaatataaaactcttatataattaaattcgcacgatgattgtatgctaaattgctaatcaattattgaaaacgaaaagggcccaagtgcccatatgcttaatttatgtacgatttaatttcctccactagcttgcatgattttaaaaccagcatacaaaccttctataaatactagcattcttcagctactaactctcatccccaacatccctcaaacaactatcagtaacatacacaccaaacaaaaacaac

SEQ ID NO: 20 - DNA, 23 bp, Artificial
  Synthesized
ataaaattcaaaaatgtcggatg

SEQ ID NO: 21 - DNA, 23 bp, Beta vulgaris
gagaaatggtggacaatactaca

SEQ ID NO: 22 - DNA, 569 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcaatattcagcagattgagggtaataataatgtgtataaaataagatttgctggggaatcagatgttggtttctttgagaaagatgggcttttgggtatcactaatgagatccctcttcctgtgtccagaagtta

SEQ ID NO: 23 - protein, 189 aa, Beta vulgaris
MASIFLKSTTTVLLLIFSTLCIATAVVIQDTDGDALRNGGQYYIIPVSAGFQGGLTLKSKAENSPCPLYITRDKVETSRGIPVTIASPYRIAIITSSIPIGIVFTNTPNVCMQPLGWQVVADEKTGQSYVATGGNGFGFNPTESFNIQQIEGNNNVYKIRFAGESDVGFFEKDGLLGITNEIPLPVSRS

SEQ ID NO: 24 - DNA, 569 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcaatattcagcagattgagggtaataataatgtgtataaaataagatttgctggggaatcagatgttggtttctttgagaaagatgggcttttgggtatcactaatgagatccctcttcctgtgtccgaagttta

SEQ ID NO: 25 - protein, 189 aa, Beta vulgaris
MASIFLKSTTTVLLLIFSTLCIATAVVIQDTDGDALRNGGQYYIIPVSAGFQGGLTLKSKAENSPCPLYITRDKVETSRGIPVTIASPYRIAIITSSIPIGIVFTNTPNVCMQPLGWQVVADEKTGQSYVATGGNGFGFNPTESFNIQQIEGNNNVYKIRFAGESDVGFFEKDGLLGITNEIPLPVSEV

SEQ ID NO: 26 - DNA, 566 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagtgagcgcaggctttcagggcggcctcaccttaaaatcaaaagccgataactcaccatgcccactctatataacccgagataaagtcgaaacgtctcgtggcatccctgttactattgcttccccttacagaatagcaatcatcacatcatctataccgataggcatagtcttcactaacactcccaacatttgtatgcagccattagggtggcaggtggttgcggatgaaaaaacaggtcagtcgtacgtcgcaacaggtggtaatggctttggatttaatcccactgagagtttcaatattcagcagattgagggtaatgataatgtgtataaaataagatttgctggggaatcagatgttggtttctttgagaaagatgggcttttgggtatcactaatgagatccctcttcctgtgtccagaag

SEQ ID NO: 27 - protein, 188 aa, Beta vulgaris
MASIFLKSTTTVLLLIFSTLCIATAVVIQDTDGDALRNGGQYYIIPVSAGFQGGLTLKSKADNSPCPLYITRDKVETSRGIPVTIASPYRIAIITSSIPIGIVFTNTPNICMQPLGWQVVADEKTGQSYVATGGNGFGFNPTESFNIQQIEGNDNVYKIRFAGESDVGFFEKDGLLGITNEIPLPVSR

SEQ ID NO: 28 - DNA, 558 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcgatattcagcagattgagggtaataataatgtgtataaaataagatttgctggggaatcagatgttggtttctttgagaaagatgggcttttgggtatcactaatgagatccctcttcctgtg

SEQ ID NO: 29 - protein, 186 aa, Beta vulgaris
MASIFLKSTTTVLLLIFSTLCIATAVVIQDTDGDALRNGGQYYIIPVSAGFQGGLTLKSKAENSPCPLYITRDKVETSRGIPVTIASPYRIAIITSSIPIGIVFTNTPNVCMQPLGWQVVADEKTGQSYVATGGNGFGFNPTESFDIQQIEGNNNVYKIRFAGESDVGFFEKDGLLGITNEIPLPV

SEQ ID NO: 30 - DNA, 558 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcaatattcagcagattgagggtaataataatgtgtataaaataagatttgctggggaatcagatgttggtttctttgagaaagatgggcttttgggtatcactaatgagatccctcttcctgtg

SEQ ID NO: 31 - protein, 186 aa, Beta vulgaris
MASIFLKSTTTVLLLIFSTLCIATAVVIQDTDGDALRNGGQYYIIPVSAGFQGGLTLKSKAENSPCPLYITRDKVETSRGIPVTIASPYRIAIITSSIPIGIVFTNTPNVCMQPLGWQVVADEKTGQSYVATGGNGFGFNPTESFNIQQIEGNNNVYKIRFAGESDVGFFEKDGLLGITNEIPLPV

SEQ ID NO: 32 - DNA, 568 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcaatattcagcagattgagggtaataataatgtgtataaaataagatttgctggggaatcagatgttggtttctttgagaaagatgggcttttgggtatcactaatgagatccctcttcctgtgtccagaagtt

SEQ ID NO: 33 - protein, 189 aa, Beta vulgaris; identical to SEQ ID NO: 23

SEQ ID NO: 34 - DNA, 550 bp, Beta vulgaris
atggcttccattttcctgaaatcaaccaccactgtgctcctcctaattttctctacactttgtattgccacagccgttgttatccaagacacagacggcgatgcactgagaaatggtggacaatactacatcatcccagttagcgcaggctttcagggcggcctcaccttaaaatcaaaagccgagaactcaccatgcccactctatataacccgagataaagtcgaaacgtctcgtggcatccctgttactattgcttccccttacagaatagcaatcatcacatcatctataccgataggcatagttttcactaacactcccaacgtttgtatgcagccattaggatggcaggtggttgcggatgaaaaaacaggtcagtcgtacgtcgcaacaggtggtaatggctttggatttaatcccactgagagtttcaatattcagcagattgagggtaataataatgtgtataaaataagatttgctggggaatcagatgttggtttctttgagaaagatgggcttttgggtatcactaatgagatccctc

SEQ ID NO: 35 - protein, 183 aa, Beta vulgaris
MASIFLKSTTTVLLLIFSTLLYATAVVIQDTDGDALRNGGQYYIIPVSAGFQGGLTLKSKAENSPCPLYITRDKVETSRGIPVTIASPYRIAIITSSIPIGIVFTNTPNVCMQPLGWQVVADEKTGQSYVATGGNGFGFNPTESFNIQQIEGNNNVYKIRFAGESDVGFFEKDGLLGITNEIP

SEQ ID NO: 36 - DNA, 360 bp, Beta vulgaris
tggcatccctgttactattgcttccccttacagaatagcaatcatcacatcatctataccgataggcatagtcttcactaacactcccaacgtttgtatgcagccattaggatggcaggtggttgcggatgaaaaaacaggtcagtcgtacgtcgcaacaggtggtaatggctttggatttaatcccactgagagtttcgatattcagcagattgagggtaataataatgtgtataaaataagatttgctggggaatcagatgttggtttctttgagaaagatgggcttttgggtatcactaatgaggtccctcttcctgttgtgttccagaaagcttttgatgttttagcgatggtcta

SEQ ID NO: 37 - DNA, 20 bp, Beta vulgaris
tggcatccctgttactattg

SEQ ID NO: 38 - DNA, 21 bp, Beta vulgaris
tagaccatcgctaaaacatca